(19) United States
(12) Patent Application Publication
(10) Pub. No.: US 2026/0191443 A1
(43) Pub. Date: Jul. 9, 2026

(54) NEEDLE-BASED APPARATUS HAVING A TEMPERATURE SENSING FUNCTION

(71) Applicant: NUTROMICS TECHNOLOGY PTY LTD, BRUNSWICK,VICTORIA (AU)

(72) Inventors: MIKEL FRASER LARSON, BRUNSWICK, VICTORIA (AU); GARRY CHAMBERS, BRUNSWICK, VICTORIA (AU); ALASTAIR MCINDOE HODGES, BRUNSWICK, VICTORIA (AU); ARTHUR SHI PIN CHIEW, BRUNSWICK, VICTORIA (AU)

(21) Appl. No.: 18/878,284

(22) PCT Filed: Jun. 22, 2023
(86) PCT No.: PCT/AU2023/050565

§ 371 Date: Dec. 23, 2024

Related U.S. Application Data
(60) Provisional application No. 63/355,702, filed on Jun. 27, 2022.
(60) Provisional application No. 63/437,398, filed on Jan. 6, 2023.

(30) Foreign Application Priority Data
May 12, 2023 (WO) PCT/AU2023/050401

(51) Int. Cl. A61B 5/1473; A61B 5/00; A61B 5/01

(52) U.S. Cl. CPC A61B 5/1473; A61B 5/01; A61B 5/685; A61B 2562/0276; A61B 2562/06

(57) ABSTRACT

An electrochemical sensor apparatus for introducing a needle electrode into the skin of a subject to contact a biological fluid or a tissue in the subject and detect a target analyte. The apparatus has a movable portion which urges the needle electrode in the subject's skin and a thermal energy sensor to determine a temperature of the biological fluid or the tissue.

### FIELD OF THE INVENTION

The present invention relates generally to an apparatus for introducing a needle into the skin of a subject, the needle being retained in situ. The needle contacts a biological fluid in the subject and detects a target analyte therein. The apparatus is configured to be simple, lightweight, and have a low profile so as to be relatively unobtrusive to the subject. Moreover, the apparatus has a temperature sensing function to improve the accuracy of target analyte detection.

### BACKGROUND TO THE INVENTION

Advances in microfabrication technology in the 1990s allowed for the large-scale production of microneedle apparatuses for use in medicine.

A single microneedle typically has a length of 150 to 1500 μm, a width of 50 to 250 μm, with a tapered tip of thickness 1 to 25 μm. In the context of a biosensor apparatus, a microneedle may be electrically conductive and function as a working electrode, a counter electrode, or a reference electrode.

As a working electrode in an electrochemical biosensor, a microneedle may be coated with a detecting element such as a redox-modified aptamer or an enzyme configured to sense a specific target analyte in a biological fluid such as interstitial fluid or blood. Typically, the biosensor is interrogated by application of an electrical potential (square wave voltammetry being an example). Peak current through the working electrode is measured, and the value used to determine the amount of analyte present about the working electrode.

The prior art discloses a number of apparatuses that insert microneedles into the skin of a subject. Such apparatuses are typically configured to facilitate application of microneedles by the subject in a non-clinical setting such as in the home. Ease of use and reproducibility are key aims of these apparatuses.

Some apparatuses are dedicated to the application of microneedles only, and once that task is completed, the apparatus is removed along with the microneedles. Other prior art apparatuses are configured to be separated from the microneedles, thereby allowing the microneedles to remain in situ in the skin for a period of time after introduction. Whilst in the skin, the microneedles may sense the rise and fall in the concentration of an analyte in interstitial fluid.

Yet a further type of prior art apparatus is configured to introduce the microneedles, with the apparatus and the microneedles remaining in situ on the subject for a period of time. While these apparatuses offer simplicity for the subject using the apparatus, they nevertheless present a number of problems.

One problem is that such apparatuses are generally obtrusive and are readily noticeable by the subject. The apparatus may catch on clothing or any other nearby object leading to complete or partial dislodgement. These apparatuses may need to be worn overnight, with significant discomfort arising where the subject rolls onto the apparatus.

A further problem is that prior art apparatuses are complex having a large number of individual parts. This increases the cost of the apparatus and also the propensity for failure. A large number of parts also increases the weight of the apparatus thereby increasing obtrusiveness for the subject. The discomfort associated with weight is found to increase proportionally with the duration for which the apparatus is worn. For some applications (such as hormone monitoring) continuous real-time data may be required over a period of weeks. While the apparatus is likely to be changed a number of times over that period, the problem of the subject wearing a weighty apparatus for an extended period remains.

A further problem arises in that the subject may be uncertain if the microneedles have properly penetrated the skin at first instance, and furthermore whether they remain properly embedded in the skin over time. Prior art apparatuses typically comprise a housing, the lower face of which sits flush on the surface of the skin. It is difficult, if not impossible, for the subject to view the surface of the skin to check for proper microneedle embedment given the presence of the housing. Where there is doubt, the apparatus may be removed and a new one applied to the skin. Replacement of apparatuses will be wasteful where the microneedles were in fact properly inserted.

In microneedle-based biosensor apparatuses such as those described above, problems arise where interaction of the target analyte with the working electrode is temperature dependent, as is often the case. For example, the conformation or structure of a detecting element may alter according to temperature, particularly where the detection element is a biomolecule such as a protein or a nucleic acid. Such changes in conformation or structure may alter the binding kinetics for the target analyte. Where the detecting element is an enzyme, the rate of catalysis may increase or decrease in response to temperature.

In any event, a calibration curve or other standard against which sensor output is assessed will not provide an accurate baseline where the standard has been established at a different temperature to that of the test sample at the time a reading is taken. Of course, an inaccurate baseline will translate into an inaccurate analyte concentration value.

The problem of temperature dependency may not arise in laboratory-based analyses where the temperature of a sample may be tightly controlled so as to be the same or similar to the temperature of the relevant standard.

However, in many applications the sample temperature cannot be controlled, and a temperature discrepancy will very likely exist between the standard and the test sample. A significant example is that where the test sample is an in situ biological fluid. While normal body temperature is generally taken to be 37.5° C., body temperature fluctuates significantly in an individual over the course of a day, with larger variations seen during an episode of infection, or even in response to environmental temperatures. Moreover, significant variations are seen between individuals tested under same conditions. It has been found that these variations are sufficient such that a standard performed at 37.5° C. will provide for an inaccurate output where the test sample is within the expected bounds of expected body temperature variation.

The prior art provides for the use of a mathematical correction factor to account for any temperature difference between a standard sample and a test sample. While these approaches are generally effective in improving sensor output accuracy, they nevertheless rely on the accurate determination of the test sample temperature. This presents a particular challenge where the test sample is an in situ biological fluid and an accurate temperature reading is not readily obtainable because the fluid is not accessible to a temperature probe.

It is an aspect of the present invention to provide an improvement to prior art microneedle-based biosensors. The improvement may be in any one or more of obtrusiveness, size, weight, complexity, cost, the ability to monitor for incorrect embedment, or accuracy. An improvement or improvements may be provided by only one embodiment of the invention. In some circumstances, the present invention may provide no improvement whatsoever and instead provide only a useful alternative to prior art apparatuses.

The discussion of documents, acts, materials, devices, articles and the like, is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

In a first aspect, but not necessarily the broadest aspect, the present invention provides an electrochemical sensor apparatus for contacting one or more projecting portions to a biological fluid or a tissue beneath the skin of a subject for an extended period, the apparatus comprising:
one or more projecting portions, each configured to penetrate the skin;
a skin contacting portion defining a skin contacting surface and one or more spaces allowing the one or more projecting portions to extend therethrough;
a movable portion configured to move the one or more projecting portions from a first position behind the skin contacting surface to a second position proud of the skin contacting surface;
optionally a retaining portion configured to, in use, retain the skin contacting surface in contact with the skin; and
a thermal energy sensor configured to determine a temperature of the biological fluid or the tissue about the one or more projecting portions when contacting the biological fluid or the tissue,
wherein the movable portion is configured to move from the first position to the second position.

In one embodiment of the first aspect, the movable portion moves in a generally arcuate path or other type of non-linear path.

In one embodiment of the first aspect, the movable portion has a connected end and a free end.

In one embodiment of the first aspect, the free end travels a greater distance than the connected end.

In one embodiment of the first aspect, the non-linear path is described by reference to the free end.

In one embodiment of the first aspect, the non-linear path is less than about 10 mm, 9 mm, 8 mm, 7 mm, 6 mm, 5 mm, 4 mm, or 3 mm.

In one embodiment of the first aspect, the degree measure of the arc is less than about 45°, 40°, 35°, 30°, 25°, 20°, 15°, 14°, 13°, 12°, 11°, 10°, 9°, 8°, 7°, 6°, or 5°.

In one embodiment of the first aspect, the movable portion has a pivoting portion, a hinging portion, a flexing portion, or an attaching portion.

In one embodiment of the first aspect, the movable portion is associated with a mounting portion.

In one embodiment of the first aspect, in use, the mounting portion is stationary, and the movable portion is movable relative to the mounting portion.

In one embodiment of the first aspect, the mounting portion comprises a portion allowing the movable portion to pivot, hinge, flex, or attach.

In one embodiment of the first aspect, the mounting portion is in fixed spaced relation to the skin contacting surface.

In one embodiment of the first aspect, the mounting portion is spaced less than about 10 mm, 9 mm, 8 mm, 7 mm, 6 mm, 5 mm, 4 mm, 3 mm, or 2 mm, from the skin contacting surface.

In one embodiment of the first aspect, the mounting portion is generally lateral to the movable portion.

In one embodiment of the first aspect, the apparatus further comprises a user actuatable releasing portion configured to retain the movable portion in the first position until user actuation of the releasing portion, at which time the movable portion is released and allowed to move to the second position.

In one embodiment of the first aspect, the apparatus further comprises a locking portion configured to lock the movable portion when in the second position.

In one embodiment of the first aspect, the apparatus is configured such that movement of the movable portion from the first position to the second position requires a motive force originating internal and/or external to the apparatus.

In one embodiment of the first aspect, the motive force internal to the apparatus originates from a spring, an elastically deformable member, a shape memory member, or other biasing means; and the motive force external to the apparatus originates from a user.

In one embodiment of the first aspect, the apparatus is devoid of an internal motive force generator configured to move the movable portion from the first position to the second position.

In one embodiment of the first aspect, the retaining portion is or comprises a dermatologically acceptable composition disposed on or about the skin contacting surface.

In one embodiment of the first aspect, the dermatologically acceptable composition is an adhesive or a functional equivalent thereof.

In one embodiment of the first aspect, the retaining portion is configured to mechanically retain the skin contacting surface in contact with the skin.

In one embodiment of the first aspect, the retaining portion is selected from any one or more of: a strap, a band, a belt, a clamp, a grip, a tie, a clasp, a sleeve, a stocking, a sock, a glove, a cap, a hat, an underpant, a singlet, a shirt, a brassiere, a top, a trouser, a scarf, a ring, a spectacle, and a choker.

In one embodiment of the first aspect, the one or more projecting portions is/are mechanically connected directly or indirectly to the moving portion.

In one embodiment of the first aspect, the one or more projecting portions is/are wire(s), needle(s), and/or microneedle(s).

In one embodiment of the first aspect, the one or more projecting portions forms an array.

In one embodiment of the first aspect, the one or more projecting portions is/are of sufficient length so as to be contactable with the epidermis, the dermis, or the hypodermis of the subject.

In one embodiment of the first aspect, the one or more projecting portions is/are configured to function, in use, so as to: conduct an electric current to or from or through the skin, conduct a sound wave to or from or through the skin, conduct light to or from or through the skin, conduct heat to or from or through the skin, sample a fluid or a tissue from the skin, or deliver a biologically active substance to the skin, or introduce an analyte sensing substance to the skin.

In one embodiment of the first aspect, the one or more projecting portions is/are each electrically conductive and the apparatus further comprises a circuit having an audio, visual or tactile indicator, the circuit configured to actuate the indicator when the one or more projecting portion(s) are in contact with an electrically conductive fluid naturally present in the skin.

In one embodiment of the first aspect, the circuit comprises at least two projecting portions and the circuit is configured to be completed by the at least two projecting portions contacting the electrically conductive fluid naturally present in the skin so as to actuate the indicator.

In one embodiment of the first aspect, the circuit comprises one projecting portion and at least one electrically conductive pad placed against the skin and the circuit is configured to be completed by the projecting portion and the pad electrically communicating with the conductive fluid naturally present in the skin so as to actuate the indicator.

In one embodiment of the first aspect, the apparatus comprises a housing dimensioned such that when the apparatus is applied to the skin and the movable portion is in the second position and any part of each of the one or more projecting portions proud of the skin contacting surface is/are embedded in the skin, the housing extends above the skin for most part or for substantially all part no more than about 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm.

In one embodiment of the first aspect, the extended period is greater than about 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 84 hours, or 96 hours.

In one embodiment of the first aspect, the apparatus is configured such that the one or more projecting portions are inseparable, or not separable without the assistance of a tool, from the apparatus.

In one embodiment of the first aspect, the movable portion and the mounting portion are integral.

In one embodiment of the first aspect, the integral moving portion and mounting portion is fabricated from an elastically deformable material.

In one embodiment of the first aspect, the integral moving portion and mounting portion is part of a circuit board of the apparatus.

In one embodiment of the first aspect, the movable portion is biased toward the second position and maintained in the first position and against the bias by the user actuatable releasing portion until actuation of the releasing portion, at which time the movable portion is released and allowed to move to the second position.

In one embodiment of the first aspect, the user actuatable releasing portion is a ledge configured to retain the movable portion in the first position, and a motive force provided by the user deforming the ledge and/or the movable portion so as to allow the moving portion to release from the ledge and move to the second position.

In one embodiment of the first aspect, the movable portion is in hinged association with the skin contacting portion.

In one embodiment of the first aspect, the hinge is disposed at or toward a peripheral region of the movable portion and the skin contacting portion.

In one embodiment of the first aspect, the releasing portion comprises a member configured to maintain the movable portion in the first position, but is removable or deformable by the user so as to allow the movable portion to move to the second position.

In one embodiment of the first aspect, the member is removable by sliding generally across the skin contacting portion.

In one embodiment of the first aspect, the member is generally wedge-shaped, and the apparatus comprises a hinge associating the movable portion with the skin contacting portion, and the thin portion of the wedge disposed proximal to the hinge and the thick portion of the wedge disposed distal to the hinge.

In one embodiment of the first aspect, the releasing portion is removable from the apparatus and comprises a gripping portion to facilitate manual removal.

In one embodiment of the first aspect or the third aspect, the one or more projecting portions have an analyte detecting element associated therewith.

In one embodiment of the first aspect or the third aspect, the one or more projecting portions are configured to function as a working electrode.

In one embodiment of the first aspect or the third aspect, the one or more projecting portions have a reference solution in electrical communication therewith and is configured to function as a reference electrode.

In one embodiment of the first aspect or the third aspect, the one or more projecting portions are configured to function as a counter electrode to a working electrode.

In one embodiment of the first aspect or the third aspect, the one or more projecting portions are dedicated to function to determine the temperature of the biological fluid or the tissue about the one or more projecting portions when inserted into the skin of the subject.

In one embodiment of the first aspect or the third aspect, the apparatus comprises a thermal insulating material configured to retain thermal energy within the one or more projecting portions.

In one embodiment of the first aspect or the third aspect, the thermal insulating material surrounds a terminal portion of the one or more projecting portions that is distal to a portion of one of the one or more projecting portions that inserts into the skin.

In one embodiment of the first aspect or the third aspect, the thermal insulating material forms a cap on a terminal portion of the one or more projecting portions that is distal to a portion of one of the one or more projecting portions that inserts into the skin.

In one embodiment of the first aspect or the third aspect, the thermal insulating material surrounds the thermal energy sensor and the one or more projecting portions.

In one embodiment of the first aspect or the third aspect, the one or more projecting portions are fabricated at least in part from a metal, a metal alloy, a combination of metals, or a ceramic.

In one embodiment of the first aspect or the third aspect, the metal, the metal alloy, a metal in the combination of metals, or the ceramic, has a thermal conductivity k of at least about 200, 300, or 400 W/mK.

In one embodiment of the first aspect or the third aspect, the metal, the metal alloy, or a metal in the combination of metals is, or comprises, any one or more of copper, steel, silver, nickel, tin, zinc, lead, aluminum, and silicon.

In one embodiment of the first aspect or the third aspect, the thermal energy sensor is configured to detect thermal energy in one of the one or more projecting portions.

In one embodiment of the first aspect or the third aspect, the thermal energy sensor is applied to or directed toward the one or more projecting portions.

In one embodiment of the first aspect or the third aspect, the thermal energy sensor contacts the one or more projecting portions, or is otherwise in thermal communication therewith.

In one embodiment of the first aspect or the third aspect, the thermal communication is via a thermally conductive flowable substance disposed between the thermal energy sensor and one of the one or more projecting portions.

In one embodiment of the first aspect or the third aspect, the thermal energy sensor is a thermocouple, a thermistor, or an infrared sensor.

In one embodiment of the first aspect or the third aspect, one of the one or more projecting portions forms part of the thermocouple or the thermistor.

In one embodiment of the first aspect or the third aspect, one of the one or more projecting portions comprises a lumen.

In one embodiment of the first aspect or the third aspect, the thermal energy sensor is disposed at least in part within the lumen or functions at least in part via the lumen.

In one embodiment of the first aspect or the third aspect, the lumen holds a thermally conductive flowable substance to form thermal communication between the thermal energy sensor and the surface of the lumen.

In one embodiment of the first aspect or the third aspect, the thermal energy sensor is configured to detect thermal energy of tissue adjacent to one of the one or more projecting portions.

In one embodiment of the first aspect or the third aspect, the thermal energy sensor is applied or directed toward to the skin surface.

In one embodiment of the first aspect or the third aspect, the thermal energy sensor contacts the skin or is otherwise in thermal communication with the skin.

In one embodiment of the first aspect or the third aspect, the thermal communication is via a thermally conductive solid material that on a first side is in thermal communication with the thermal energy sensor and on a second side is in thermal communication with the skin.

In one embodiment of the first aspect or the third aspect, the thermally conductive solid material is a metal or a plastic, or a plastic with a filler.

In one embodiment of the first aspect or the third aspect, the filler is graphite, graphene, carbon fibre, or other carbon-based material.

In one embodiment of the first aspect or the third aspect, the thermally conductive solid material has a thickness of less than about 5 mm, 4 mm, 3 mm, 2 mm, 1 mm, 0.9 mm, 0.8 mm, 0.7 mm, 0.6 mm, 0.5 mm, 0.4 mm, 0.3 mm, 0.2 mm, or 0.1 mm.

In one embodiment of the first aspect or the third aspect, the thermal energy sensor is a thermocouple, a thermistor, or an infrared sensor.

In one embodiment of the first aspect or the third aspect, the one or more projecting portions and the thermal energy sensor are contained within, or otherwise associated with a housing.

In one embodiment of the first aspect or the third aspect, the analyte detecting element is an aptamer.

In one embodiment of the first aspect or the third aspect, comprising a redox active species in association with the aptamer and configured so as to function to report interaction of a target analyte with the aptamer.

In a second aspect, the present invention provides a method for contacting one or more projecting portions of an electrochemical sensor apparatus to a biological fluid or a tissue beneath the skin of a subject, the method comprising the step of providing the apparatus of any embodiment of the first aspect, contacting the skin contacting surface of the apparatus to the subject, and causing or allowing the movable portion to move from the first position to the second position, and causing or allowing the thermal energy sensor to determine a temperature of the biological fluid or the tissue.

In one embodiment of the second aspect, the apparatus remains contacted to the skin for a period of greater than about 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 84 hours, or 96 hours.

In a third aspect, the present invention provides an electrochemical sensor apparatus for contacting one or more projecting portions to a biological fluid or a tissue beneath the skin of a subject, the apparatus comprising:
one or more projecting portions, each configured to penetrate the skin;
a skin contacting portion defining a skin contacting surface and one or more spaces allowing the one or more projecting portions to extend therethrough;
a movable portion configured to move the one or more projecting portions from a first position behind the skin contacting surface to a second position proud of the skin contacting surface;
a retaining portion configured to, in use, retain the skin contacting surface in contact with the skin;
a user actuatable releasing portion configured to retain the movable portion in the first position until user actuation of the releasing portion, at which time the movable portion is released, and caused or allowed to move to the second position; and
a thermal energy sensor configured to determine a temperature of the biological fluid or the tissue about the one or more projecting portions when contacting the biological fluid or the tissue.

In one embodiment of the third aspect, the releasing portion comprises a member configured to maintain the movable portion in the first position, but is removable or deformable by the user so as to allow the movable portion to move to the second position.

In one embodiment of the third aspect, the member is removable by sliding generally across the skin contacting portion.

In one embodiment of the third aspect, the member is generally wedge-shaped, and the apparatus comprises a hinge associating the movable portion with the skin contacting portion, and the thin portion of the wedge disposed proximal to the hinge and the thick portion of the wedge disposed distal to the hinge.

In one embodiment of the third aspect, the releasing portion is removable from the apparatus and comprises a gripping portion to facilitate manual removal.

In a fourth aspect, the present invention provides a method for contacting one or more projecting portions to a biological fluid or a tissue beneath the skin of a subject, the method comprising the step of providing the apparatus of any embodiment of the third aspect, contacting the skin contacting surface of the apparatus to a subject, and actuating the user actuatable releasing portion so as to cause or allow the movable portion to move from the first position to the second position, and causing or allowing the thermal energy sensor to determine a temperature of the biological fluid or the tissue.

### BRIEF DESCRIPTION OF THE FIGURES

FIG. 1 illustrates highly diagrammatically and in lateral view a microneedle embedding apparatus of the present invention. The embodiment relies on a biasing means to provide a motive force for insertion of the microneedles into the skin. The arm is shown in the first position (20a), as it is presented to the user, and in the second position (20b) when the microneedles are embedded in the skin. The curvature in the movable arm is shown deliberately exaggerated to better demonstrate the operation of the embodiment as a whole. While such a curvature will be operable (and therefore not excluded from the ambit of the invention), the curvature will typically be of a materially lower magnitude.

FIG. 2A illustrates highly diagrammatically and in lateral view a further microneedle embedding apparatus of the present invention. The embodiment relies on the user to provide the motive force for insertion of the microneedles into the skin. The arm is shown in the first position (205a), as it is presented to the user, and in the second position (205b) when the microneedles are embedded in the skin.

FIG. 2B illustrates a variation of the embodiment of FIG. 2A, being devoid of an upper housing.

FIG. 3A illustrates an upper perspective view of an embodiment of the present invention that utilises a printed circuit board (PCB) as the biasing means to provide the motive force for insertion of the microneedles into the skin. The arm (20) is shown in the first position as it is presented to the user, and before embedment of the microneedles into the skin.

FIG. 3B illustrates the embodiment of FIG. 3A, but in lower perspective view.

FIG. 4 illustrates an upper perspective view a microneedle embedding apparatus of the present invention. The embodiment relies on the user to provide the motive force for insertion of the microneedles into the skin. The arm is shown in the first position as it is presented to the user, and before embedment of the microneedles in the skin.

FIG. 5A illustrates a lower perspective view of the embodiment of FIG. 4.

FIG. 5B illustrates an upper perspective view of the embodiment of FIG. 4.

FIG. 6 illustrates a lower perspective view of the embodiment of FIG. 4 more completely showing the removable flexible layer that is removed to expose the dermatologically acceptable adhesive.

FIG. 7 illustrates in lower perspective view the microneedle embedding apparatus of FIG. 4 having the removable flexible layer removed to expose the dermatologically acceptable adhesive.

FIG. 8 illustrates in lower perspective view the microneedle embedding apparatus of FIG. 7 with the microneedles in an extended position, as required for embedment in the skin of a subject.

FIG. 9 illustrates a further microneedle apparatus of the present invention comprising a temperature sensor. The apparatus is further configured to prevent the outward extension of the microneedles until the apparatus is applied to the skin surface. The central area of the drawing sheet shows the components of the apparatus in lateral view, and in exploded form. Each component is shown in perspective view in the peripheral areas of the drawing sheet.

FIG. 10 illustrates diagrammatically and in cross-section, a needle of the present invention, having an associated temperature sensor in the form of a thermistor or thermocouple making contact with the needle.

FIG. 11 illustrates diagrammatically and in cross-section, a needle of the present invention, having an associated temperature sensor in the form of an infrared sensor module directed toward the needle.

FIG. 12 illustrates diagrammatically and in cross-section, a needle of the present invention, having an associated temperature sensor in the form of a thermistor or thermocouple making contact with the needle, and an insulating cap enshrouding the needle and temperature sensor.

FIG. 13 illustrates diagrammatically and in cross-section, a needle of the present invention, having an associated temperature sensor in the form of an infrared sensor module directed toward the needle, and an insulating cap enshrouding the needle albeit with a window formed therein to allow exposure of a target surface of the needle.

FIG. 14 illustrates diagrammatically and in cross-section, a needle of the present invention, having a lumen within which a temperature sensor in the form of a thermocouple or a thermistor is disposed.

FIG. 15 illustrates diagrammatically and in cross-section, a needle of the present invention having a lumen, the needle functioning as a first metal in a thermocouple and a needle extending through the lumen to function as a second metal in the thermocouple.

FIG. 16 illustrates diagrammatically and in cross-section, a wearable apparatus comprising an electrochemical sensor and a temperature sensor.

FIG. 17 illustrates diagrammatically a basic circuit of an electrochemical sensor having a temperature sensor providing input into a processor configured to determine a target analyte concentration.

Unless otherwise indicated herein, features of the drawings labelled with the same numeral are taken to be the same features, or at least functionally similar features, when used across different drawings.

The drawings are not prepared to any particular scale or dimension and are not presented as being a completely accurate presentation of the various embodiments.

### DETAILED DESCRIPTION OF THE INVENTION AND PREFERRED EMBODIMENTS THEREOF

After considering this description it will be apparent to one skilled in the art how the invention is implemented in various alternative embodiments and alternative applications. However, although various embodiments of the present invention will be described herein, it is understood that these embodiments are presented by way of example only, and not limitation. As such, this description of various alternative embodiments should not be construed to limit the scope or breadth of the present invention. Furthermore, statements of advantages or other aspects apply to specific exemplary embodiments, and not necessarily to all embodiments, or indeed any embodiment covered by the claims.

Throughout the description and the claims of this specification the word “comprise” and variations of the word, such as “comprising” and “comprises” is not intended to exclude other additives, components, integers, or steps.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment, but may.

As used herein, positional terms such as “lateral”, “across”, “above”, “over” “below”, “higher”, “lower”, “upward”, “downward”, “plan view”, and the like, are to be considered with reference to an apparatus of the invention when applied to an upwardly facing area of the skin of a subject, such as the upper surface of a human thigh when the human is seated on a chair. It will be understood that the apparatus may be applied to an area of skin having a different orientation to the upright orientation just defined in which case the skilled person is amply enabled to reformulate the aforementioned positional terms.

The term “subject” is used to refer to an animal (including a human and a non-human animal) to which the present apparatus may be applied. The term “user” is used to refer to a human that applies the apparatus to a human or a non-human animal. The subject and the user may be the same human subject, but not necessarily so.

A “biological fluid” may be any biological fluid of a subject, including but not limited to, interstitial fluid, blood, saliva, a lacrimal secretion, a lactational secretion, a nasal secretion, a tracheal secretion, a bronchial secretion, an alveolar secretion, a gastric secretion, a gastric content, a glandular secretion, a vaginal secretion, a uterine secretion, a prostate secretion, semen, urine, sweat, cerebrospinal fluid, a glomerular filtrate, an hepatic secretion, bile, or an exudate, any of which are contacted in use with a needle electrode of the electrochemical sensor in vivo. A “tissue” includes a volume comprising one or more cells.

Unless the contrary intention is apparent from the context of use, the terms “needle”, “microneedle” and “wire” are used interchangeably. Each is functionally the same or similar, being able to insert into the skin of a subject to contact a biological fluid or a tissue.

Various distinct embodiments of the invention are disclosed herein (whether by way of drawing or written description), the embodiments having one or more features disclosed in context thereof. It will be appreciated that there is no intention to limit the application of a certain feature or combination of features to use in the embodiment in which it is disclosed. For example, a first embodiment may be disclosed to comprise features A and B, with a second embodiment disclosed to comprise features C and D. The intention is to include within the ambit of the present invention an embodiment having any one, two, three or four of the features A, B, C and D in any available combination.

It will, however, be apparent to the skilled person that certain combinations are less preferred or indeed contraindicated. For example, where feature B requires feature A to operate, and embodiment comprising the combination of features B, C and D may be unworkable.

The present invention is predicated at least in part on the finding that an improved or alternative sensor apparatus is provided where a temperature sensing function is provided to improve the accuracy of an output of the apparatus. Further details of the temperature sensing function are provided infra.

The apparatus comprises a moveable portion that urges the microneedles into the subject's skin, travels in a non-linear path. Moreover, the non-linear path may be of a limited length and where the path is arcuate of a limited degree measure. By this arrangement, a major moving part of the apparatus requires only a limited range of motion in the vertical direction to bear on and insert the microneedles into the subject's skin. The limited range of motion allows for the housing of the apparatus to assume a relatively low profile when viewed in the lateral direction. Thus, the apparatus rises to a relatively small height over the skin and is therefore less obtrusive to the subject.

Moreover, the non-linear path of the movable portion allows for a simplified mechanism to be used. For example, the movable portion may move by way of a simple flexing or hinging mechanism. These mechanisms require a relatively small number of components, allowing for overall a smaller, lighter, simpler, more reliable, and less expensive apparatus to be developed.

Certain embodiments of the invention have further features, which either when taken alone or in combination with other features provide further advantage or a further useful alternative to the prior art. Such embodiments will be more fully described by reference to the non-limiting preferred embodiments described infra.

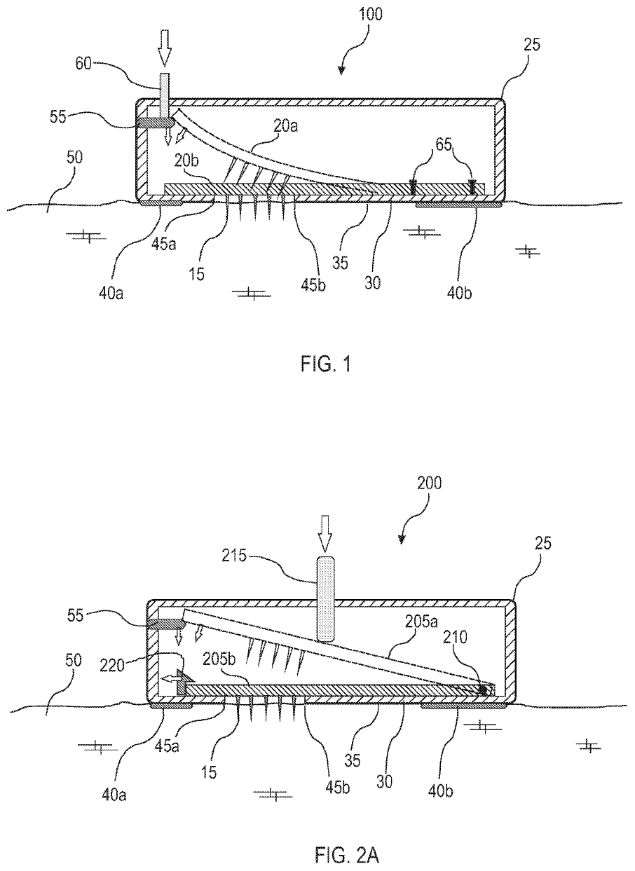

Reference is made to FIG. 1 showing a basic form of the present apparatus (10) having a microneedle array (one microneedle marked 15) attached to movable portion, which in this embodiment is an elastically deformable arm (20). The arm (20) is biased to assume a linear configuration (20b), however is initially presented to the user with the arm flexed into an upward curvature as shown in the dashed representation (20a).

The apparatus (10) comprises a rigid housing (25) having a skin contacting portion (30) on its lower side which defines a downwardly facing skin contacting surface (35). The surface (35) is placed onto the subject's skin, and is retained therein by areas of a dermatologically acceptable adhesive (40a, 40b). A suitable adhesive will typically be capable of resisting water to allow the subject to bathe normally. The adhesive will typically have sufficient adhesion to inhibit detachment that may arise in the course of everyday activities such as dressing, undressing, sleeping, performing domestic chores, light to moderate intensity sporting activities, brushing past objects while walking and the like. The level of adhesive is typically not so great so as to cause any difficulty, unpleasant sensation, pain, irritation, or skin damage in removing the apparatus.

An exemplary adhesive is a synthetic rubber adhesive or tackified acrylic adhesive of the type used on medical tapes. A double-sided medical tape may be used, such as 3M™ 1577 tape, with one side adhering to the apparatus and the other adhering to the subject's skin.

The skin contacting portion (30) comprises a space (45), the margins of which are marked (45a) and (45b). The spaces (45) provide respective passages through which the microneedles (15) pass, allowing the terminal regions of the microneedles to penetrate and embed into the underlying skin (50) when the arm (20) is in the linear position (20b).

The arm (20) is retained in its flexed state by the ledge (55) which functions as a releasing means. When the user wishes to insert the microneedles (15) into the skin (50) they depress the button (60) as shown by the arrow. The lower face of the button (60) bears on the ledge (55), and because the ledge (55) has some ability to deform (being fabricated from a rubber-like material, or formed from a flexible projection of the inner face of the housing (25) for example) it bends downwardly under the force so as to release the edge of arm (20a). The elastic nature of the arm (20a) causes it to rapidly return to its biased linear position (20b) thereby forcing the microneedles (15) into the underlying skin (50). The The ledge (55) is configured so as to exhibit sufficient resilience to resist the biasing force in the arm (20a) however that resilience is not sufficient to resist the downward force exerted by the button (55) when depressed.

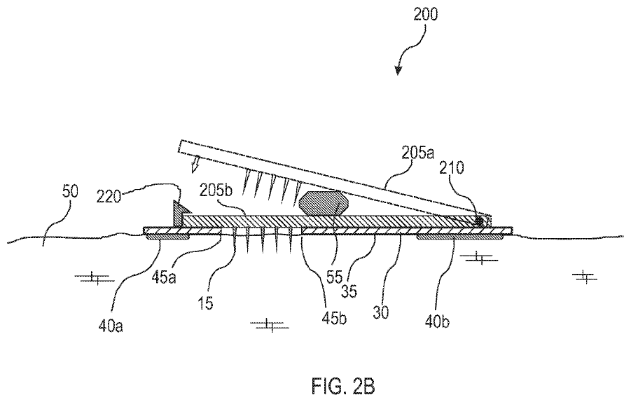

In the embodiment of FIG. 1, the arm (20) is fixed at one end to the housing (25) by the fasteners (65). While the arm (20) is flexible, the flexibility is not so high so as to easily move away from position (20b) when in place on the skin (50) of the subject. As will be appreciated, any movement of the arm (20) away from position (20b) may cause the microneedles (15) to withdraw from skin (50). Given the bias of the arm (20) toward the position (20b) there may be no need for a locking mechanism to maintain the arm in position (20b). However, if required a suitable locking mechanism is described infra for the embodiment of FIG. 2A.

FIG. 2A shows an alternative basic form of the apparatus (200) whereby the arm (205) is rigid and hinged to the housing (25) by way of hinge pin (210). The embodiment of FIG. 2A operates similarly to that of FIG. 1 so far as a ledge (55) acts as a releasing means. However, in the embodiment of FIG. 2A the button (215) acts on the rigid arm (205a). The rigid arm (205) transfers the force of the button to the deformable ledge (55) causing the ledge (55) to bend and therefore release the free end of the arm (205a). The button (215) continues to be depressed by the user until the arm assumes the position (205b), and in which position the microneedles (215) are embedded into the skin (50). Again, a point on the free end of the arm (205a) travels along a non-linear path, and in this embodiment the path is an arc that is a segment of a circle, the origin of the circle being at the hinge pin (210).

It will be appreciated that the hinged arrangement of the FIG. 2A embodiment provides no resistance to the arm (205) hinging away from the position (205b) while the apparatus is being worn. A danger that the microneedles (15) withdraw from the skin (50) whilst in situ therefore presents. Accordingly, a locking mechanism is provided to maintain the arm in position (205b). The mechanism comprises a deformable latch (220), being fabricated from a material with some flexibility or from an internal projection formed from the housing (25) material, for example. The latch (220) has a sloped upper face, and upon contact with the rigid arm (205) the entire latch (220) is forced to bend to the left (as drawn) under the force being applied by the user via the button (215) and the sloped upper face. Once the terminus of the arm (205) clears the lower corner of the sloped upper face, the latch (220) resumes its normal upright position (as drawn) and the free end of the arm (205b) seats securely in the recess at the base of the latch (220).

An alternative to the embodiment of FIG. 2A is shown at FIG. 2B. In FIG. 2B, the apparatus (200) is devoid of an upper housing. The arm (205a) is maintained in position by the releasing means (55), which is removable by the user in this embodiment when the apparatus (200) is applied to the subject. After removal of the releasing means (55) the arm (205a) is depressed downwardly by the user so as to assume the second position (205b).

It will be noted in the embodiments of FIG. 1, FIG. 2A and FIG. 2B that when released from the ledge (55), the free end of the arm (20 or 205) moves in a non-linear manner as it returns to its biased position (20b). If a single point on the free end of the arm (20 or 205) is considered, that point travels along a non-linear path which describes an arc. In the context of the present invention, the terms “arc”, “arcuate”, and similar terms refer to a curve joining any two points. The term “arc” should not be interpreted restrictively to mean only a segment of a circle, although in some embodiments it is a segment of a circle (see the embodiment of FIG. 2A for example).

As will be clear from the basic embodiments of both FIG. 1, and FIGS. 2A and 2B, in each case the arm (20 or 205) travels a relatively small distance when transitioning from the first position to the second position. Indeed, in these embodiments (and certain other embodiments) the apparatus is deliberately configured such that the arm is not able to travel along any path that is outside that between the first and second positions. Put another way, the apparatus may be configured such that the arm is unable to travel along any path that is outside the shortest distance between the first and second positions.

By placing limits on the path along which the arm may travel, advantage is provided in so far as the height of the apparatus (in the vertical direction, as drawn) is also limited. Accordingly, the apparatus may assume a low profile (in a dimensional sense) extending above the subject's skin a relatively short distance.

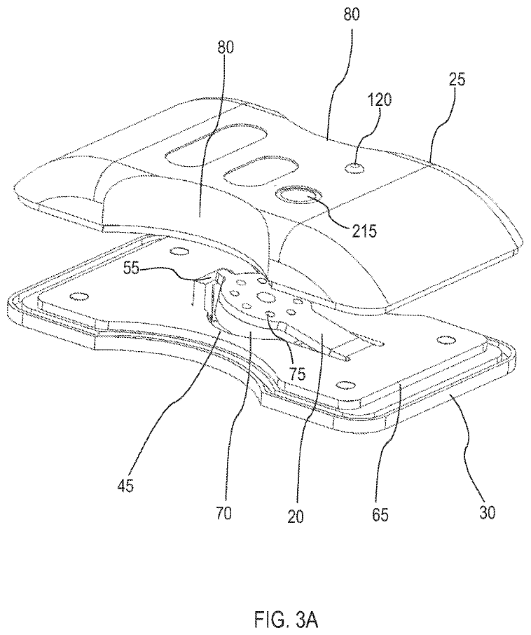

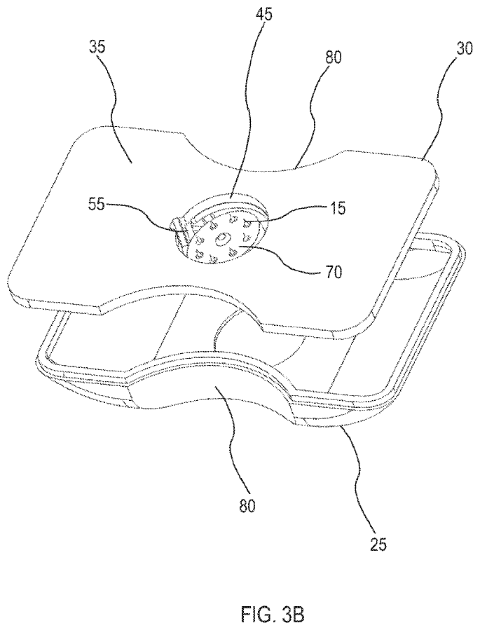

Turning now to FIG. 3A and FIG. 3B, there is shown a preferred apparatus constructed generally in accordance, and operable generally consistently with, the embodiment of FIG. 1. The arm (20) is formed integrally with a PCB (65) carrying the various electronic components required for operation of the apparatus. The PCB material is elastically deformable allowing the arm (with attached microneedles at the terminals) to flex upwardly as drawn to position the arm in the first position, but when released to assume the second position due to the natural bias in the arm toward the second position.

The arm (20) is maintained in the first position by the arm (20) terminus resting on the ledge (55) as shown most clearly in FIG. 1A. In this position, the microneedles (15) are retained within the apparatus with no part extending through the spaces (45). This is the configuration in which the apparatus is provided for use, and in which it is applied to the subject's skin.

The arm (20) has connected thereto a microneedle mounting block (70) supporting the microneedles. The mounting block (70) also contains conduits (not drawn) to carry electrical current from each of the microneedles (15) to one of a number of connection points (75) of the PCB (65). By this arrangement, electrical signals may be conveyed to and/or from microneedles embedded in the subject's skin. For example, the apparatus may be configured as a sensor with the microneedles configured to contact a biological fluid or a tissue in the subject's body to detect an analyte therein. The biological fluid may be, but is not limited to, interstitial fluid, blood, or a mixture thereof. The electrical signals from the microneedles are conveyed to the PCB for amplification, filtering, encoding, analysis, transmission, or any other electrical or electronic process.

In this embodiment, the PCB serves the dual function of carrying the apparatus electronics and also as motive means for moving the microneedles from a position internal the apparatus to an external position. The PCB material has been found to be well suited to providing the limited range of motion preferred for the arm of the present apparatus. By this arrangement, the number of components in the apparatus is lessened.

The upper face of the housing (25) reveals the actuating surface of a button (215) which is depressible by the finger of a user. The button (215) is biased upwardly (as drawn) by a spring, or due to it being formed integrally with the housing (25) material. In the latter form of biasing, the button (215) may be mounted on an arm which is integral with the housing material and biased such that the upper surface of the button (215) is coplanar with the housing (25).

A lower portion (not visible) of the button (215) bears on the upper surface of the arm (20), the upper surface being the rear surface of the PCB (65) such that depression of the button (215) urges the arm (20) downwardly so as to release from the ledge (55) and assume the second position. In the second position, it will be appreciated that the microneedles will extend through respective spaces (45) and embed into the underlying skin (e.g., the epidermis, the dermis, or the hypodermis of the subject).

The natural bias of the PCB (65) material toward the second position is sufficiently strong for the arm (20) to remain in the second position without the need for any means of locking the arm in the second position. Accordingly, microneedles (15) are able to remain embedded in the subject's skin for an extended period.

In an alternative embodiment, the arm (20) has a curved configuration when in the second position, and is naturally biased away from the second position. In another embodiment, the bias of the arm (20) toward the second position is not sufficiently strong so as to prevent any movement away from the second position. In such embodiments (and other embodiments) a locking mechanism may be provided to prevent movement of the arm away from the second position such that the microneedles (15) do not retract into the apparatus and remain embedded in the skin. A suitable locking mechanism is the latch mechanism as disclosed in relation to other embodiments herein. Other locking mechanisms will be apparent to the skilled person having the benefit of the present specification.

The housing (25) comprises opposed depressions (80) to facilitate gripping between the user's thumb and second finger, and holding the apparatus against the skin's surface. The user's first finger is free to actuate the button (215) so as to embed the microneedles (15) into the underlying skin.

The skin contacting surface (35) may have a dermatologically acceptable adhesive layer (not drawn) applied thereto so as to maintain the apparatus in situ on the subject's skin for an extended period. The adhesive layer can cover a portion or substantially all of the skin contacting surface (35). A manually releasable flexible layer may cover the adhesive until the apparatus is to be applied to the skin, as described for other embodiments of the apparatus as described herein.

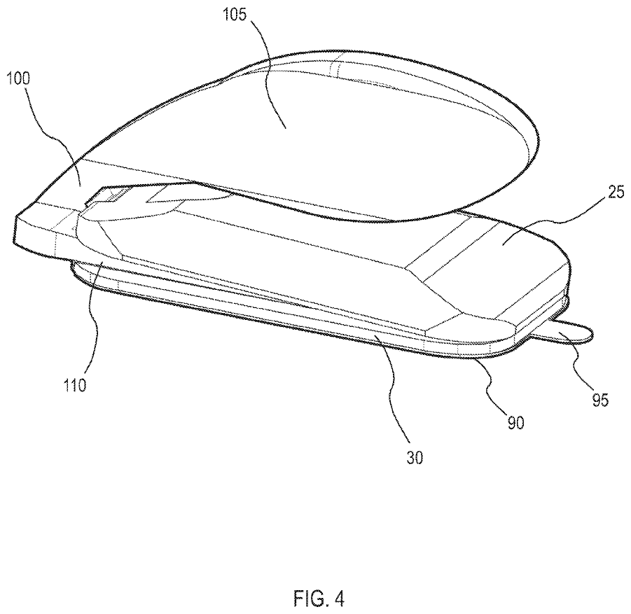

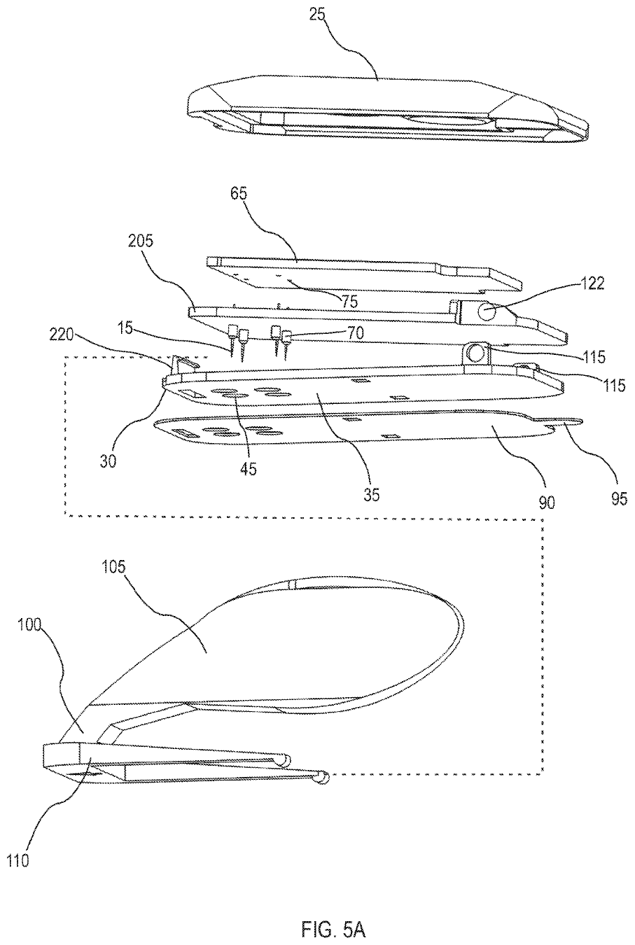

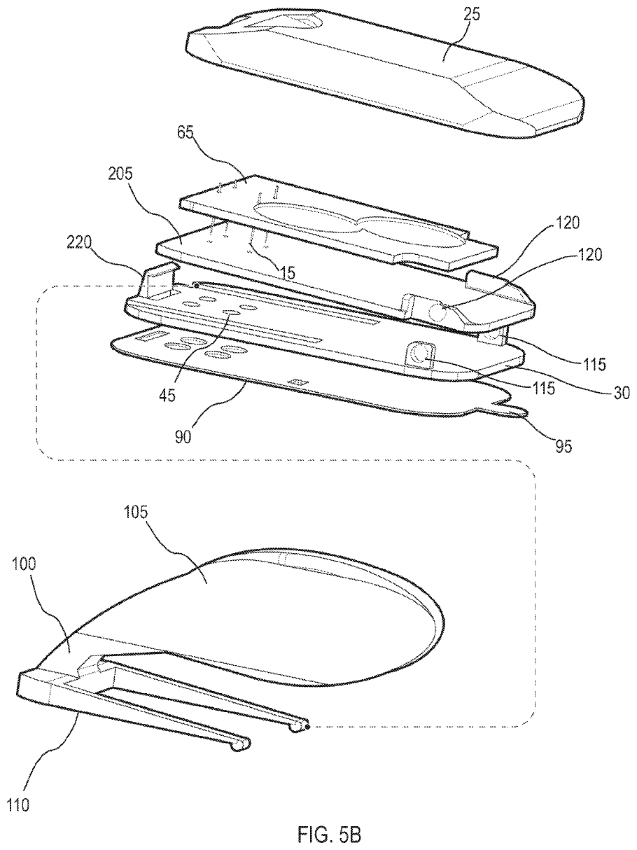

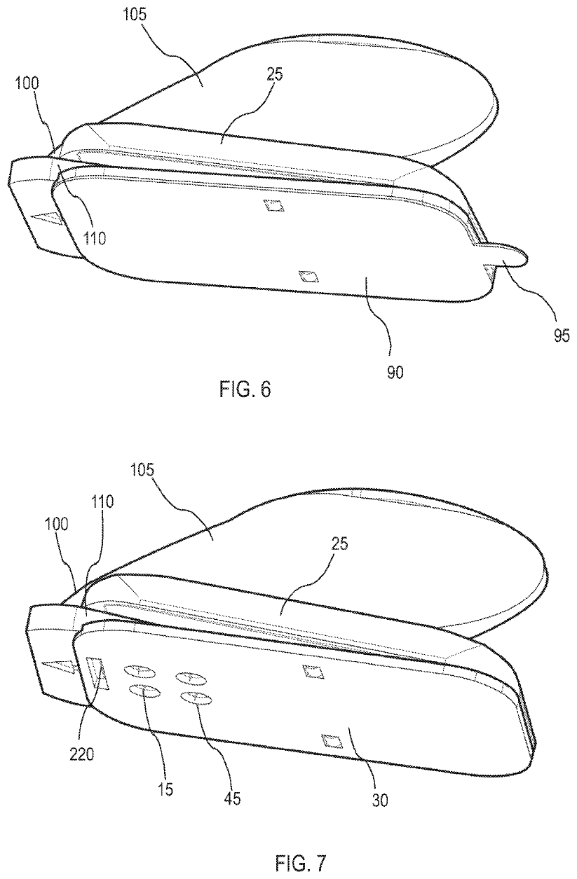

Turning now to FIG. 4, FIG. 5A, FIG. 5B, FIG. 6, and FIG. 7, there is shown a preferred apparatus constructed generally in accordance and operable generally consistently with the embodiment of FIG. 2B.

The embodiment comprises an upper housing portion (25) and a skin contacting portion (30). Also provided is a removable flexible layer (90) being graspable by way of the tab (95), the removal of which exposes a dermatologically acceptable adhesive on the skin contacting surface (35). As explained supra, the adhesive is for the purpose of retaining the apparatus on the subject's skin for an extended period. The flexible layer (90) functions to prevent curing or drying of the adhesive, prevent contamination of the adhesive layer before use and/or premature attachment of the adhesive to packaging, or to other surfaces. In a particularly preferred embodiment, in addition to covering the adhesive layer, the flexible layer (90) extends over the spaces (45) to prevent contamination of the microneedles (15) and also help prevent unintended needle-stick injuries to a user.

The apparatus may have a retaining portion functioning to retain the apparatus on the skin such that the projecting portions remain in contact with a biological fluid or a tissue of the subject. The retaining portion may be dedicated to that function, or may perform another function.

In many circumstances, a retaining portion being or comprising a dermatologically acceptable adhesive will be useful. Adhesives allow for simplicity in application of the apparatus by a user, often requiring only the removal of a protective backing sheet to expose the adhesive and then contacting the exposed adhesive to the skin. This method of application is similar to the application of a sticking plaster, and is therefore already a familiar process to users.

As an alternative to the use of adhesives, the retaining portion may be some mechanical means for maintaining the apparatus in the required position on the skin. For example, the apparatus may comprise a dedicated strap that engages about limb that is adjustable so as to keep the apparatus firmly applied to the subject. As an alternative, the apparatus may be incorporated into a wearable item such as a glove or a shirt, or an item of jewellery such as a ring which functions to retain the apparatus in position. The apparatus may be configured to engage with a discrete wearable item (such as by complimentary hook-and-loop means), or may have the wearable item integral therewith.

In some embodiments, the apparatus is retained simply by the wearable item bearing against the housing. For example, the retaining portion may be a snug-fitting elasticised glove which is worn over the apparatus.

In some embodiments, the retaining portion is any surface or part of the apparatus which contacts the skin of the subject, with a feature of the subject being at least partially responsible for maintaining the apparatus in place on the subject. For example, the apparatus may be configured to be retained between two parts of the body normally in close apposition, or within an existing anatomical structure. The apparatus may be shaped and/or dimensioned to be retained between the toes, the buttocks, in the groin, in the buccal cavity, in a nostril, in the ear canal, or in the umbilicus.

In other embodiments the apparatus housing is shaped and/or dimensioned to snugly fit over a digit, a toe, or an ear, for example. The apparatus housing may be elastically deformable, composed of a rubberised material for example, and configured to be stretched over any anatomical part (such as a finger).

Each of the aforementioned embodiments is considered to be a retaining portion in the context of the present invention.

The apparatus further comprises a releasing member (100) having a grasping portion (105) and a wedging portion (110), the function of which will be more fully described infra.

Turning now to the exploded views of FIG. 5A and FIG. 5B, components that are analogous to those in earlier figures will be immediately apparent.

In this embodiment, the motive force responsible for moving the arm (205) thereby urging the microneedles (15) into the underlying skin is provided by the user. In use, the user places a finger on the upper housing (25) and pushes downwardly. Furthermore, the arm (205) is movable by way of a hinging arrangement.

The hinging arrangement is provided by way of opposing lugs (115) extending from skin contacting portion (30), each lug comprising an aperture. The arm (205) comprises opposing laterally extending discs (120), each of which seats into an aperture of the lugs (115). It will be apparent that the arm (205) is able to hinge relative to skin contacting portion (30) to allow movement from the first position to the second position.

The arm (205) is presented to the user having the arm in the first position. The arm (205) is maintained in the first position by the wedging portion (110) of the releasing member (100). Before removal of the releasing member (100) the wedging portion inserts between the skin contacting portion (30) and the arm (205), thereby keeping the microneedles within the apparatus.

When intending to apply the apparatus to the subject's skin, the user removes the flexible layer (90) by pulling on the tab (95) to expose the adhesive layer on the skin contacting surface (35). The apparatus is then applied to the skin, with the adhesive maintaining it in situ for an extended period.

Once the apparatus has been applied to the skin, the user grasps the grasping portion (105) and pulls laterally to the left (as drawn), so as to completely remove the releasing member (100). The releasing member (100) has no further function and is discarded at this juncture. By removal of the releasing member (100) the arm (205) is released from the first position and permitted to move (under a downward force exerted by the user) into the second position whereby the lower face of arm (205) contacts the upper face of the skin contacting portion (30). In the second position, the microneedles (15) extend through the spaces (45) and into the underlying skin.

As will be appreciated, the releasing member (100) may be configured to prevent the upper housing (25) of the apparatus from closing to the skin contacting portion (30) when not intended by the user. The releasing member (100) is inserted or otherwise juxtaposed between the upper housing (25) and the skin contacting portion (30) to prevent closure of the upper housing (25) towards the skin contacting portion (30) sufficient to allow the tips of the microneedles (i.e., projecting portions) to protrude from the base of the holes in the skin contacting portion (30). Preventing closure also prevents movement of the arm (205) from the first position to the second position. Thus, when the releasing member (100) is in place, the tips of the microneedles cannot be inadvertently accessed to cause microneedle contamination or injury. In using the apparatus, the user removes the releasing member (100) as a step in the use process. In a preferred embodiment of apparatus use, the user first adheres the apparatus to the subject's skin and then removes the releasing member (100), prior to pressing the upper housing (25) to insert the microneedles into the skin.

Prior to removal by the user, the releasing member (100) can be kept in place by any one of a variety of features. In one example the releasing member (100) comprises protrusions that fit into recesses in either the upper housing (25), the skin contacting portion (30) or both the upper housing (25) and the skin contacting portion (30) to assist in retaining it in place until intentionally removed. In another example the releasing member (100) is designed to be slidably assembled to the skin contacting portion (30) or upper housing (25), such that friction between the releasing member (100) and either the upper housing (25) or the skin contacting portion (30) assists in keeping it in place until intentionally removed. In yet another example magnetic force may be used to assist in keeping the releasing member (100) in place. In a one embodiment of the invention, a magnet mounted within the releasing member (100) is positioned so as to be proximal to a Hall effect sensor positioned in either the upper housing (25) or the skin contacting portion (30), when the releasing member (100) is in place. According to this embodiment, when the releasing member (100) is removed by the user, the Hall effect sensor detects the removal of the magnet and causes the apparatus to take some action, such as powering up the electronic circuitry ready for use, converting it from sleep mode to active mode. It is to be understood that the above are examples of possible methods for assisting in retaining the releasing member (100) in place prior to intentional removal that may be used alone or in combination and that other methods as known in the art can also be used alone or in combination with the examples given.

In some embodiments of the invention, the releasing member (100) can also function as a covering element that is used to cover the microneedles after the apparatus has been removed from the subject. In a preferred example of this embodiment the locking element is located on the upper housing (25), extending down towards the skin contacting portion (30). The releasing member (100) comprises a groove that allows the releasing member (100) to slide past the locking element when the releasing member (100) is being withdrawn from the apparatus, while keeping the face of the releasing member (100) facing the upper surface of the skin contacting portion (30) continuous. In use, a releasing member (100) according to this preferred embodiment is removed by the user prior to pressing the upper housing (25) to insert the microneedles into the subject's skin and retained by the user. After the apparatus is removed from the subject post use, the user is instructed to adhere the releasing member (100) to the adhesive layer on the lower surface of the skin contacting portion (30) to cover the protruding microneedles. In another example of this embodiment, the releasing member (100) is flexibly attached to the apparatus such that the releasing member (100) can remain attached to the apparatus after it has been withdrawn by the user and then repositioned to cover the protruding microneedles after the apparatus has been removed from the subject post use. In yet another example of this embodiment, the releasing member (100) and the upper housing (25) are designed such that the releasing member (100) can be slidably or otherwise engaged with the upper housing (25) once it has been removed, where it is intended that the releasing member (100) be stored while the apparatus is in use and removed to be used as a covering element after the apparatus has been removed from the subject.

In some embodiments, of the apparatus is configured to facilitate the user in removing the apparatus from the subject. As will be appreciated, the use of an adhesive layer may result in difficulty in removal of the apparatus from the skin. Examples of such configuration include leaving a portion of the skin contacting surface (35) uncoated with adhesive, such that a gap is present between the subject's skin and the surface (35), wherein the user uses the gap as a leverage point to assist in pulling the apparatus away from the skin by breaking the adhesive bond. In another example, a leverage mechanism not located on the skin contacting surface is incorporated to allow a taller gap than that created by the absence of adhesive on a portion of the skin contacting surface. In yet another example, a tab extending beyond at least one edge of the skin contacting portion (30) and attached to the adhesive layer can be incorporated, where the user pulls on the tab with sufficient force to cause the adhesive layer to stretch and yield, further causing the adhesive to delaminate from the skin contacting surface (35) and the skin.

In some embodiments of the invention, the apparatus is designed such that the releasing member (100) is locked into place in its position prior to apparatus use unless pressure is applied to the upper housing (25). This embodiment is intended to further ameliorate the risk of the releasing member (100) being prematurely withdrawn. In an example of this embodiment, there are features on the releasing member (100) and on at least one of the upper housing (25) and skin contacting portion (30) that are lockably engaged when the upper housing (25) is not being pressed. When the upper housing (25) is depressed, the feature on at least one of the upper housing (25) and skin contacting portion (30) is distorted, so as to disengage the releasing member (100) and allow it to be withdrawn.

In yet other embodiments, the releasing member (100) need not be removed from the apparatus by the user. According to these embodiments, the releasing member (100) comprises a flexible element of sufficiently high stiffness that it does not substantially deflect when subjected to closing forces likely to be present on the apparatus during manufacture, storage and in the user's hands prior to application to the subject, but flexible enough that it deflects when the user intentionally applies a closing force to the apparatus when it is applied to the subject's skin. In so flexing, the releasing member (100) is deflected, allowing the upper housing (25) to close towards the skin contacting portion (30). In these embodiments, the releasing member (100) could also function as the locking element, or the releasing member (100) could be separate from a locking portion. In some of these embodiments, a feature such as that labelled as (220) in FIG. 5A, FIG. 5B, and FIG. 7, forms the releasing member (100).

Each space (45) of the apparatus is dimensioned such that a microneedle can extend through it clearly, with at least a tapered part of the microneedle not impacting the sides of the hole during insertion. In some embodiments the holes may be of sufficient cross-section such that no part of the microneedle will contact the sides of the space during insertion. In other embodiments, at least a part of the hole along its length will have a cross-section such that a portion of the length of the microneedle contacts the sides of the hole during insertion. According to this embodiment the hole functions to help support a portion of the length of the microneedle to assist in preventing bending of the microneedle as it is inserted.

In some embodiments of the apparatus, the skin contacting portion (30) comprises further spaces or depressions configured to accept protrusions on the releasing member, to assist in retaining the releasing member until it is removed by the user. In addition, or alternatively, the skin contacting portion (30) comprises protrusions designed to be accepted into recesses in the releasing member to assist in retaining the releasing member in place until deliberate removal by the user.

The embodiment depicted in FIG. 4, FIG. 5A, FIG. 5B, FIG. 6, and FIG. 7, comprises a locking portion in the form of a latch (220) which permanently locks the arm (205) in the second position preventing the arm (205) from any hinging movement. In the drawn embodiment, the latch (220) is a simple unitary member capable of deflecting in response to movement of the arm (205) toward the closed position, but then returning to its original position when the arm (205) is in the second position (205b), thereby locking the arm (205) in place.

Rather than act on the arm (205), the locking portion may act on another component of the apparatus, that component in turn locking the arm in place. For example, the locking portion may act on the upper housing (25), with the upper housing (25) in turn retaining the arm (205) in the second position. In a further alternative the locking portion may act on the PCB (65), with the PCB (65) in turn retaining the arm (205) in the second position.

In other embodiments, the locking portion comprises a recess into which a protrusion on the upper housing (25) is inserted to lock the upper housing (25) in a closed position (i.e., with the arm (205) in the second position). In one embodiment, the locking portion comprises a flexible element that is designed to allow the locking portion to move when impinged upon by the upper housing (25), so at to allow the housing (25) to close relative to the skin contacting portion (30) and whereby once the upper housing (25) has closed, allows the locking portion to move to lock in place the upper housing (25) in the closed position. In one embodiment, the apparatus comprises a protrusion on the upper housing (25), designed to be inserted into a recess in the locking portion, the protrusion comprising a flexible element to allow the protrusion to move, allowing the upper housing (25) to close relative to the skin contacting portion (30) and whereafter the housing (25) has closed relative to the skin contacting portion (30) the protrusion moves to be inserted in the recess in the locking portion, so as to lock the upper housing (25) in the closed position. The flexible element may comprise a shaft that is sufficiently deformable to allow the upper housing (25) to close without yielding of the shaft, so that the flexible element will try to return to its original position post the upper housing (25) closing. In a less preferred, but nonetheless functional embodiment, the flexible element comprises a coil spring.

A flexible element of the locking portion may be fabricated from any suitable material having the necessary stiffness and yield point. Examples of suitable material include non-crystalline plastics, crystalline plastics, sprung steel, unsprung steel, stainless steel, or other materials as are known if the art with suitable mechanical properties.

In a preferred embodiment of the invention, the locking portion is fabricated from the same material as the skin contacting portion (30), to facilitate the fabrication of a skin contacting portion with an integral locking portion.

In a particularly preferred embodiment of the invention, the force required to deflect or otherwise move the flexible element is designed to be large enough that the pressure the user needs to supply to deform the flexible element and thus cause the upper housing (25) to close towards the skin contacting portion, is sufficient to insert the microneedles into the skin. According to this embodiment, the flexible element of the locking portion is used to set the force necessary to close the apparatus (thereby causing the arm to assume the second position) and ensure that the force is sufficient to insert the microneedles in their intended position embedded in the skin.

In other embodiments, the locking portion comprises at least one adhesive region located on at least one of the lower surface of the upper housing (25) and the upper surface of the skin contacting surface (35). When the apparatus is closed, the one or more adhesive regions adhere the upper housing (25) to the skin contacting portion (30), locking the apparatus in the closed position.

In another embodiment of the invention, the locking portion can assume three different stable states. In a first state, the locking portion is in a disengaged configuration, before the upper housing (25) is pushed downwardly towards the skin contacting portion (30) to close the apparatus. In a second state, the locking portion is in a first engaged position. When the locking portion is in the first engaged position it serves to lock the microneedles (15) in the embedded position in the skin (i.e., the arm (205) being in the second position). In a third state, the locking portion is in a second engaged position. In this state, the locking portion locks the apparatus in the open position (i.e., with the arm (205) in the first position) with the microneedles withdrawn into the apparatus to ameliorate the possibility of needle-stick injury resulting from microneedles protruding after apparatus use. In an example of this embodiment, the locking portion comprises a user engagement portion, that can be gripped or otherwise engaged by the user, for example by engaging a fingernail under an overhanging ledge, so that the user can deflect the flexible portion of the locking portion. According to this example, to close the apparatus the user presses on the upper housing (25) and locks it in place, as in other embodiments disclosed herein. When it is desired to remove the apparatus from the subject, the user engages with the locking portion and deflects it in a first direction, so as to unlock the upper housing (25) from the skin contacting portion (25), and then deflect the locking portion in a second direction, to lock the apparatus in the open position (i.e., with the arm in the first position) with the microneedles in the withdrawn position. In a preferred embodiment of this example, in the first direction, the locking portion is moved away from the body of the apparatus, and in the second direction, is moved towards the body of the apparatus. When deflected sufficiently in the second direction, the locking portion is designed, for example, to be stably engaged in a recess so as to prevent closure of the apparatus without intentionally doing so.

In some embodiments of the invention, a downward force on the microneedles when inserted into the skin is provided via the flexible element of the locking portion applying a downward force when the apparatus is locked in the closed position (i.e., with the movable arm in the second position). In some embodiments, effective locking of the movable arm in the second position is provided by a dedicated spring or other suitable biasing means. In other embodiments, the spiring or other biasing means is not dedicated to a locking function and may, for example, act also as a motive force in the movement of the arm from the first position to the second position. For example, a torsion spring may apply a closing torque at a pivot point (where present). In yet another example a flat, disk or coil spring is mounted to the rear of microneedles, such that when the apparatus is closed the spring is distorted or compressed so as to apply a downward force on the microneedles when the apparatus is in the closed position.

Although not an essential feature of the invention, the PCB (65) will be required for many applications where the microneedles are for the purpose of conducting electrical current to, from or through the skin. In that regard, the PCB may carry a microprocessor, and/or volatile electronic memory (such as RAM) and/or non-volatile electronic memory (such as ROM) and/or a wireless networking module (such as a Bluetooth™ module). The apparatus will of course comprise a power source, typically by way of a button battery.

The embodiment depicted in FIG. 3A further comprises a light emitting diode (LED) (120) viewable by the user. One function of the LED (120) may be to confirm to the user and/or the subject that the microneedles are properly embedded in the skin at application, and remain so for the extended period of wear.

The LED makes electrical connection with the PCB (65), which in turn makes electrical connection with the microneedles (15). Proper embedment of the microneedles can be determined by reference to any one of more of current flow, resistance to current, or impedance between two microneedles.

Alternatively, proper embedment of a single microneedle can be determined by reference to any one of more of current flow, resistance to current, or impedance between the single microneedle and some other electrical contact of the apparatus with the skin. As an example, an electrically conductive pad can be placed against the surface of the skin, where in some examples the conductive pad is placed on the face of the housing that contacts the skin. This electrically conductive pad in concert with at least one of the microneedles complete an electrical circuit when the microneedle is inserted into the skin. Completion of this circuit is used to indicate correction insertion of the microneedles.

The electronics involved may be simple, any example of which being a biological fluid of the skin, such as interstitial fluid (which is naturally conductive) acts to complete a circuit including the LED. The assumption is made that proper embedment is indicated by the simple contact of a microneedle with a biological fluid. The LED is illuminated where the microneedle contacts the biological fluid (or vice versa) thereby providing a visual indication of correct embedment.

More sophisticated electronic arrangements may be required to provide a higher level of assurance of correct microneedle embedment. For example, regard may be had to whether or not a minimum length of microneedle is embedded, thereby providing an assurance of insertion of the microneedle to a certain minimum depth. The apparatus may comprise electronic means of measuring the quantum of a parameter such as current flow, with a higher current flow being indicative or more complete embedment of a microneedle. Program instructions executed by a processor on-board or otherwise associated with the apparatus may use as input a parameter such as current flow (possibly in conjunction with other physiological or environmental parameters) to provide an indication of the degree of embedment of the microneedle.

A further function of the LED may be to provide other information such as battery charge level. For example, the LED may be connected to a microprocessor capable of monitoring battery voltage, with the microprocessor causing the LED to blink red when voltage falls below a predetermined threshold value. That value may be a voltage that is somewhat above the minimum operating voltage to allow the subject time to access a replacement battery (or replacement apparatus where the battery is not user-serviceable) before the apparatus becomes inoperable.

In other embodiments, the LED may produce an output indication of a data connection status. For example, the LED may blink alternating red and green light to warn of a disruption in a wireless data connection with a remote device such as a smartphone. A smartphone may be responsible for processing sensor output, and warning the subject by an audible output when a threshold (such as glucose concentration) is breached. In such embodiments, the LED and an apparatus networking module may be connected to a microprocessor, the microprocessor monitoring the connection status of the module and causing the LED to produce an output when the connection is made and/or lost. While application software on the smartphone may be configured to alert the subject to a loss of data connection, the smartphone may lose power (by running out of charge, for example) and in which case the only means by which the subject could be alerted is by way of the apparatus itself.

Similar output functions to the LED may be provided by a buzzer or a miniature speaker to provide audio output comprehensible by the subject. The output may a tone, a series of tones, or a synthesised voice for example.

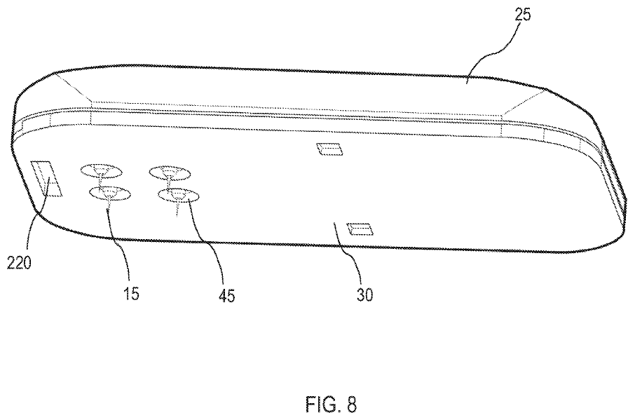

Reference is now made to an alternative embodiment of the apparatus shown at FIG. 9, being a modified version of the embodiment depicted in FIG. 4 through to FIG. 8. The embodiment of FIG. 9 includes a temperature sensor (300) which in operation extends through the space (305) in the skin contacting portion (30) so as to contact the surface of the subject's skin. The temperature sensor (300) may be a thermocouple or a thermistor, for example, in operable connection with a microprocessor on the PCB (65). The temperature sensor may directly contact the skin, or may be separated from the skin by way of a thermally conductive material.

The temperature sensor may be disposed within a pocket or other formation dimensioned to receive the temperature sensor. The pocket may be fabricated from a thin sheet-like material of a plastic, such as a thermally conductive plastic having a metal or other filler to facilitate transmittance of thermal energy from the underlying skin to the temperature sensor. The temperature sensor may be surrounded by a thermally conductive paste to facilitate transfer of thermal energy from the pocket wall to the temperature sensor.

The floor of the pocket may extend outwardly from the apparatus such that the floor of the pocket is pushed gently onto the skin surface when the apparatus is applied thereto, thereby facilitating transfer of thermal energy from the skin to the temperature sensor. It will be understood that overly firm pushing of the pocket floor onto the skin surface may force blood out the skin capillaries thereby artificially cooling the skin surface.

Preferably, only the floor of the pocket is fabricated from a thermally conductive material, with the remainder being fabricated from a material of low thermal conductivity. By that arrangement, thermal energy from the skin will not be routed away from the temperature sensor.

An insulating material may form a ceiling of the pocket to ensure thermal energy is retained about the temperature sensor and not lost to the internal cavity of the housing.

The pocket may comprise a space extending through the floor so that the temperature sensor can directly contact the skin surface. A temperature that is closer to the actual skin temperature would be expected given that thermal energy is not required to traverse any intervening material.

In a further modification, the temperature sensor may be an infrared sensor module, and in which case the material of at least the pocket floor should not substantively interfere with its operation. It is contemplated that a space could be formed in the floor to allow the infrared sensor module direct exposure to the skin surface to effect an accurate reading of skin temperature.

Signal output from the temperature sensor (300) may be used in calculations made by the microprocessor (or a remote microprocessor) to more accurately determine the concentration of a target analyte. For example, the microprocessor may have access to a range of stored calibrations curves, each curve having been performed at a given temperature. Based on the output of the temperature sensor (300), the appropriate calibration curve may be selected, and a more accurate analyte concentration therefore determined.

The embodiment of FIG. 9 comprises a releasing member (100) having paired protrusions (a first protrusion marked (310), the second of the paired protrusions being obscured by the first). The protrusions (310) extend downwardly and through the spaces (315) in the skin contacting portion (30). The function of the protrusions (310) is to prevent lateral movement of the releasing member (100) until the lower face of the skin contacting portion (30) is pressed against the skin. The act of pressing against the skin causes the protrusions (310) to vertically exit the spaces (315) so as to allow the releasing member (100) to be pulled laterally away by the subject. This mechanism prevents the releasing member (30) from being inadvertently removed before the apparatus is properly applied to the skin surface. Absent such a mechanism, the microneedles (15) may be caused to prematurely extend through the spaces (45) and may become contaminated by contact with the air or an object, or become physically damaged by catching on clothing for example.

Some embodiments of the apparatus may require the upper regions of the microneedle to be electrically insulated to avoid the moist surface of the skin (as distinct from a biological fluid thereunder) forming a conducting path between microneedles.

As another means of controlling moisture, an absorptive material may be positioned on a microneedle mounting portion and proximal to the microneedle tips. In embodiments of the apparatus for sensing applications, the material is configured to absorb any excess fluid that may be produced by insertion of the microneedles in the skin to improve subject experience and to ameliorate any issues fluid contact with other parts of the apparatus, such as the electronic circuitry or electrical contacts, may cause. In embodiments of the apparatus such as fluid extraction applications, the material acts as a wicking agent to transport the fluid from the microneedle site to the required final site on the apparatus or external to the apparatus. In some embodiments the absorptive material is in the form of a sheet. In embodiments where it is desirable to prevent contamination or damage to the microneedles prior to insertion, the sheet comprises holes through which the microneedles pass, wherein the holes are dimensioned to be sufficiently large to prevent the absorptive material coming into contact with the microneedles during the microneedle insertion process, but sufficiently small to allow excess fluid exuding from the access penetration point created by a microneedle to contact and be absorbed by the material. In other embodiments, such as when the apparatus is intended to be used for fluid extraction, there are either no holes in the sheet of absorptive material, or the holes are dimensioned so that the absorptive material contacts the microneedle during and post insertion to aid in its wicking action. In the embodiment with no holes in the sheet the microneedles create holes when they pass through the sheet as part of the insertion process.

The present apparatus may be configured for use and/or used in any suitable application where microneedles are required to be embedded in a subject's skin for an extended time period.

Such applications include electrochemical aptamer-based sensing whereby a target analyte in a biological fluid or a tissue is detected by binding to a capture entity such as an aptamer comprising a redox reporter. The capture entity may be covalently or non-covalently bound to the microneedles, with the redox reporter causing an electrical signal to be conveyed by the microneedles upon binding of the target analyte. The target analyte may be a drug or other exogenous species, or an endogenous species such as a hormone or a metabolite.

Where the microneedles function as electrodes to detect analytes present in the layers of the skin, the apparatus may comprise circuitry and components to excite the electrodes electrically and to receive, measure and process the electrical signals that result from the electrical excitation. According to this embodiment the microneedles may comprise a tip, a shaft, and a base, where electrical signals are generated at electrodes either coated on to the surface of or integral to the microneedle, transmitted along the shaft of the microneedle to the base of the microneedle, where electrical connection is made to the base or shaft of the microneedle to transmit the electrical signals to and from the electrodes to the electronic circuitry. The electrodes can be formed proximal to the tip of the microneedle, on at least a portion of the shaft of the microneedle and not proximal to the tip of the microneedle or both proximal to the tip of the microneedle and on at least a portion of the shaft of the microneedle.

The microneedles may be connected to the electronic circuitry by a variety of methods as are known in the art, for example, soldering, wire wrapping or sprung loaded pins. In one embodiment, the microneedles are mounted so as to pass through a plate or a block of dielectric material with the connection portion of the microneedles positioned at or above the surface of the plate or block distal to the microneedle tips. A zebra strip connection may be used to connect the microneedles to the electronic circuitry to facilitate robust connections without the need to precisely align the zebra connector with the microneedle ends, as least in one dimension.

A further potential application is for the delivery of electrical current to the skin for the purpose of muscle stimulation, or for the stimulation or inhibitions of a biological process of the subject. Similarly, the present apparatus may be used to detect electrical currents in the subject's skin, for example to detect nerve conductance.

In any of the above applications, the microneedles may be solid or hollow, as required or as desired.

Microneedle length may be selected according to a particular application. Typically, the microneedles will be required to extend at least below the stratum corneum. The depth of the stratum corneum varies according to location, that layer being relatively thick on the soles of the feet and relatively thin on the backs of the hands, for example. Accordingly, the length of microneedle extending beyond the housing may be adjusted according to the intended site of application.

In some cases, the microneedles may be required to extend well below the stratum corneum, and into the lower layers of the epidermis, the dermis and even the hypodermis, including the subcutaneous tissue. Again, the length of the microneedles extending beyond the apparatus may be set accordingly.

The skilled person will also appreciate the possible need to set microneedle length according to the intended subject. For example, relatively short microneedles will generally be required to effect contact with the subcutaneous tissue of a neonate subject, while for the same site an adult subject will require longer microneedles

It may be desirable in some applications for one microneedle to penetrate more deeply into the skin as compared to another microneedle. The two microneedles may therefore terminate at different distances from the skin surface, or at different distances from a microneedle mounting portion. In some embodiments, the two microneedles are different lengths. In other embodiments the microneedles are the same length, and a mounting portion is configured so as to axially displace one microneedle relative to the other. For example, the mounting portion may be multi-levelled with a first electrode extending from a first level and a second electrode extending from a second level.

For typical applications, the microneedles may extend outwardly from the apparatus for a distance of between about 10 μm and about 5000 μm. For many applications, distances between about 500 μm and about 4000 μm will be useful.

Those skilled in the art will appreciate that the invention described herein is susceptible to further variations and modifications other than those specifically described.

For example, the movable arm may be moved by the user squeezing or pressing on a flexible portion of the apparatus housing, by the actuation of a rotating lever, or by sliding an element along an inclined to urge the arm downward.

The skin contacting portion of the apparatus has been drawn as being strictly planar on its underside (the skin contacting surface), however in some embodiments it may be curved to conform to the surface of a bodily part such as the finger, wrist, heel, or ear. The skin contacting portion may have a degree of flexibility (in at least one direction) so as to be conformable to the surface of a bodily part.

The space through which a microneedle extends is generally shown as being an aperture, however other types of spaces are contemplated. In some embodiments the space is not an aperture, one such embodiment having microneedles extending through a space peripheral to the skin contact portion.

The present apparatus may comprise a thermal energy sensor which is maintained on the subject in the same way as the projecting portions (such as needles) of the electrochemical sensor apparatus. The thermal energy sensor is therefore able to monitor temperature over an extended period for which a target analyte is detected. Thus, the ability to determine temperature over the extended period results in more accurate analyte concentration determination at any point in time, and also potentially over the extended period.

The inventors propose that a projecting portion (such as a needle) of an electrochemical sensor apparatus needle may perform three functions, each of the functions working together to provide an accurate determination of the concentration of a target analyte in a subject. Firstly, the needle punctures the skin and extends into the subcutaneous tissue of the subject such that a terminal portion contacts a biological fluid or tissue of the subject. Secondly, the needle terminal portion may be loaded with a detecting element (such as an aptamer) capable of selectively interacting with a target analyte such that the needle functions as a whole as a working electrode. Thirdly, the needle may function in sensing the temperature of the biological fluid or tissue of the subject. An accurate determination of the biological fluid or the tissue temperature allows for a temperature-based correction factor to be applied to a concentration of target analyte determined by the electrochemical sensor.

Needles may be fabricated from metal, silicon, polymer, glass, or ceramic, with the base of the needle typically being attached to a base substrate to form an array. The needle base substrate may comprise an adhesive to improve engagement with the skin.

The needle may, of course, be of larger dimension. The dimensions generally having no upper limit except to the extent that it must be acceptable to the subject.

The needle, when in the form of a wire, may be useful in the context of the present invention. However, wires are generally flexible and accordingly may not be sufficiently robust so as to breach the skin surface. If necessary, an introducer may be used to facilitate insertion into the subcutaneous tissue.

The electrochemical sensor of the present invention may be embodied as an electrochemical aptamer-based (EAB) sensor. The EAB sensor may be provided in the form of a wearable patch or similar having needles which extend through the skin surface and into a biological fluid or tissue of the subject in which the analyte is detectable.

An EAB sensor may be of the potentiometric, amperometric or conductometric type. In a potentiometric sensor, a local equilibrium is established at the sensor interface, where either the electrode or membrane potential is measured, and information about a sample is derived from a potential difference between two electrodes. Amperometric sensors rely on a potential being applied between a reference and a working electrode, so as to cause the oxidation or reduction of a redox-active species; with the resultant current being measured. Conductometric sensors rely on the measurement of conductivity at a series of frequencies.

EAB sensors are typically of the amperometric type, with the aptamer (such as DNA, RNA or XNA) being bound to the working electrode. Gold is often used as the probe surface for the working electrode. The aptamer has an associated redox-active species which acts as a reporter. The redox reporter is often methylene blue. Upon target analyte binding, the aptamer undergoes a conformational change, bringing the redox reporter more proximal to the working electrode surface. This increase in proximity increases electron transfer from the redox reporter to the electrode. The increase in speed of electron transfer contributes to a change in Faradaic current that is detected by a potentiostat.

Aptamers are small (usually from 20 to 60 nucleotides) single-stranded RNA, DNA or XNA oligonucleotides able to bind a target drug with high affinity and specificity. Aptamers may be considered as nucleotide analogues of antibodies, but aptamer production is an in vitro cell-free process that is significantly easier and cheaper than the production of antibodies by cell culture or in vivo methods.

Aptamers are usually selected from combinatorial library having a vast number (up to 10¹⁸) of different oligonucleotides. While RNA aptamers provide a significantly greater structural diversity compared to DNA aptamers, their application is complicated by stability issues in the presence of RNases, high temperature and unfavourable pH.

Selection of an aptamer that is selective for a given drug may be facilitated by a process known as SELEX (systematic evolution of ligands by exponential enrichment). The process may be considered as two alternating stages. In the first stage, the library oligonucleotides are amplified by a polymerase chain reaction (PCR) to the desired concentration. For the selection of RNA aptamers, the single-chained oligoribonucleotides are generated by in vitro transcription of double-stranded DNA with T7 RNA-polymerase. For DNA aptamers, a pool of single-stranded oligodeoxyribonucleotides is generated by strand separation of double-stranded PCR products. In the second stage, the products of amplification are incubated with target drug and oligonucleotides which bind the drug are used in the next SELEX round.

Separation of oligonucleotides with higher affinity for target drug and removal of unbound oligonucleotides are achieved through intense competition for binding sites. The selection pressure rises with every SELEX round. Maximum enrichment of the oligonucleotide pool with aptamers with the strongest affinity for the target molecule is usually achieved after 5 to 15 rounds.

EAB sensors are typically incorporated into a circuit having a reference electrode. The reference electrode is the site of a known chemical reaction that has a known redox potential. For example, a reference electrode based on the silver-silver chloride (Ag|AgCl) redox pair has a fixed and known potential forming the point against which the redox potential of the working electrode is measured. Also typically included in the circuit is a counter electrode which functions as a cathode or an anode to the working electrode. Because the applied voltage bias does not pass through the reference electrode (due to an impedance of the potentiostat), any potential generated is attributed to the working electrode. Current is measured as potential of the interrogating electrode versus the stable potential of the reference electrode. The difference in potential produces the current in the circuit thereby generating an output signal. The signal quantifies target binding depending on electron transfer that is ideally stoichiometrically proportional to target binding.

As discussed above, EAB sensors may be embodied in many forms, one of which is needle-based patch. When the patch is applied to a subject, the needles penetrate the subject's skin contacting a fluid of the patient. The tip of the needle functions as the working electrode, with the redox reporter tagged aptamer being associated with the tip. This arrangement provides a minimally invasive platform for real-time, continuous in vivo drug detection, which is sufficiently sensitive and selective for monitoring the amount of the drug in the body of a patient over time. EAB sensors are also capable of making single point measurements.

Aptamers and needles may be exploited together in the form of an EAB biosensor, whereby an aptamer-loaded needle is inserted through the skin so as to contact a biological fluid. The needle functions essentially as a working electrode which detects analyte in the biological fluid. Typically, a second needle is used as a counter electrode, with a third needle functions as a reference electrode.

Each aptamer molecule has an associated redox reporter such as methylene blue. Binding of the target analyte causes a conformational change in the aptamer, which in turn modulates the proximity of the redox reporter to the surface of the needle causing an increase or decrease in electron exchange with the needle surface. The resultant current changes through the needle may be used to determine the concentration of target analyte in the biological fluid.

EAB sensors have been reported to successfully detect endogenous analytes such as metabolites, hormones, antibodies, and cancer markers in a biological fluid. Exogenous analytes such as drugs, toxins, infectious agents have also been detected.

While EAB sensors show significant promise, the art recognises problems arise in terms of the accuracy of target analyte concentrations provided. Analyte concentration is typically determined by comparing the sensor output current with a calibration curve. Almost always, the calibration curve is prepared at a temperature different to that of the subject's subcutaneous tissues. To overcome that discrepancy a temperature correction factor is applied to the biosensor output. Of course, any temperature correction factor will be dependent on the accurate determination of the subject's temperature.

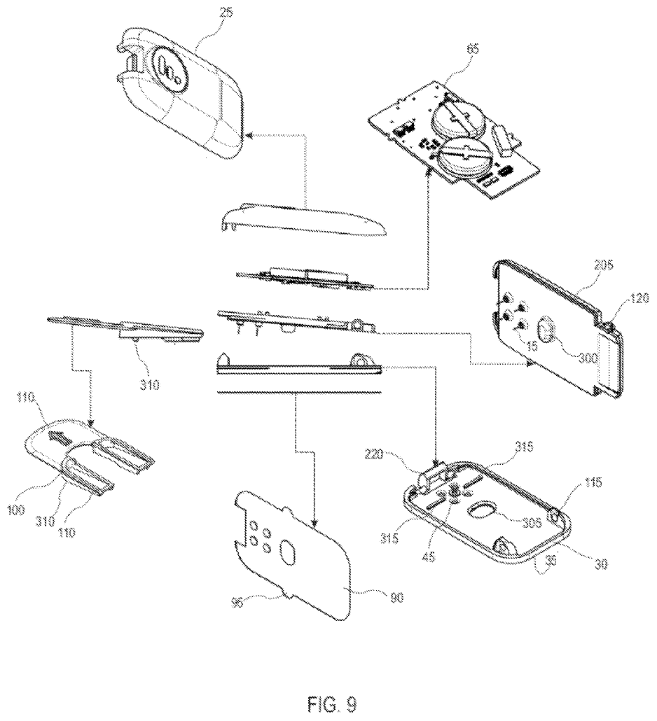

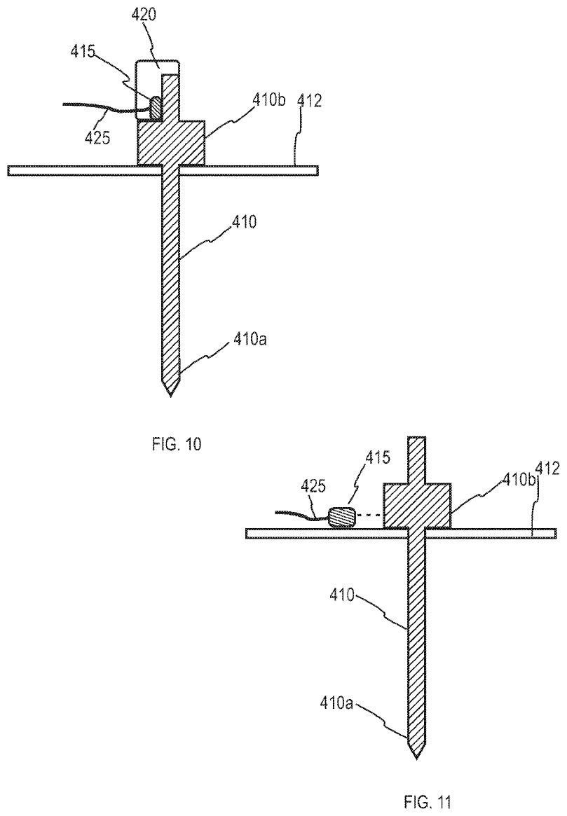

Reference is made to FIG. 10, showing an analyte and temperature sensing needle (410). The terminal portion (410a) is coated with an aptamer specific for a target analyte, the aptamer having an associated redox reporter. The needle (410) functions therefore as a working electrode. The needle (410) is mounted on a support (412), typically with a second needle (not drawn, functioning as counter electrode) and a third needle (not drawn, functioning as reference electrode).

The needle (410) is in contact with a temperature sensor (415) being, for example, a miniature thermistor or a thermocouple. A useful high accuracy small glass bead thermistor is model S14A10310 (Sensor Scientific Inc., NJ, USA). The bead dimensions are 0.36×0.5 mm allowing for incorporation with a needle having a lumen, and providing for a very low thermal mass. The low thermal mass provides a rapid response to temperature changes and minimises any active extraction of the thermal energy from the subject's tissue. Lower accuracies (if acceptable in the context of the application) are provided with the less expensive S14A10310 model. Where a thermistor contacts the subject's tissue, it may be necessary to ensure biocompatibility of the materials.

A thermally conductive material (420) such as a paste, putty, grease or gel, is applied about the needle (410) and the temperature sensor (415) so as to ensure efficient transfer of thermal energy from the needle (410) to the temperature sensor (415). A suitable paste is DP-200-30 (Taica Corporation, Japan), which is thermally conductive and electrically insulating. A useful putty is TG-NSP35-1LB (T-Global Technology Limited, United Kingdom).

The needle (410) comprises an expanded portion (410b), the upper surface of which may form an ancillary surface contactable by the temperature sensor (415) to improve thermal energy transfer. As will be understood, it is generally desirable that as much thermal energy as possible is transferred from the subcutaneous tissue of the subject to the temperature sensor (415) via the needle (410) such that the sensor (410) can be used accurately detect temperature of the tissue.

The temperature sensor (415) has paired wires (425) to carry output signal typically to a processor (not drawn), the signal being used as an input value and analysed according to program instructions.

Preferably, the sensor (415), paired wires (425), expanded portion (410b) and thermally conductive material (420) shown in FIG. 10 are dimensioned as small as possible so as to avoid any acting as a heat sink and thereby limiting the amount of thermal energy which the sensor (415) is able to absorb and report to the processor. In that regard, the support (412) is preferably fabricated from a material having low thermal conductivity, such as a plastic so as to not route thermal energy away from the temperature sensor (415).

Reference is now made to the embodiment of FIG. 11 that uses a non-contact means for measuring the temperature of the needle (410). Particularly, the temperature sensor (415) is an infrared sensor module configured to receive thermal energy (i.e., energy in the infrared spectrum). The infrared sensor module (415) may be of the active type (whereby the module both directs infrared radiation onto the target and detects infrared radiation emitted by the target) or the passive type (whereby the module only detects infrared radiation emitted by the target).

The infrared sensor module (415) may require a relatively large target area for operation, and the expanded portion (410b) of the needle may be dimensioned sufficiently to function as the target surface. In some embodiments, the expanded portion (410b) may be expanded to a greater extent than shown in the drawing so far as necessary to function as target area for the infrared sensor module (415).

Non-contact methods of temperature sensing may be preferred given that heat sinking due to the thermal masses of a thermistor, thermocouple, paste, and output wires is removed.

Reference is now made to FIG. 12, showing a modification of the embodiment of FIG. 11 to include a thermal insulation cap (430) enshrouding upper portions of the needle (410), the temperature sensor (415) and the thermally conductive material (420). The thermal insulation cap may be fabricated from a foam or similar material, and functions to inhibit the loss of thermal energy to the atmosphere. In the embodiment of FIG. 12, the thermally conductive material (420) and insulation cap (430) work together to channel thermal energy as far as possible to the temperature sensor (415) so as to properly represent the temperature in the subject's subcutaneous tissue.

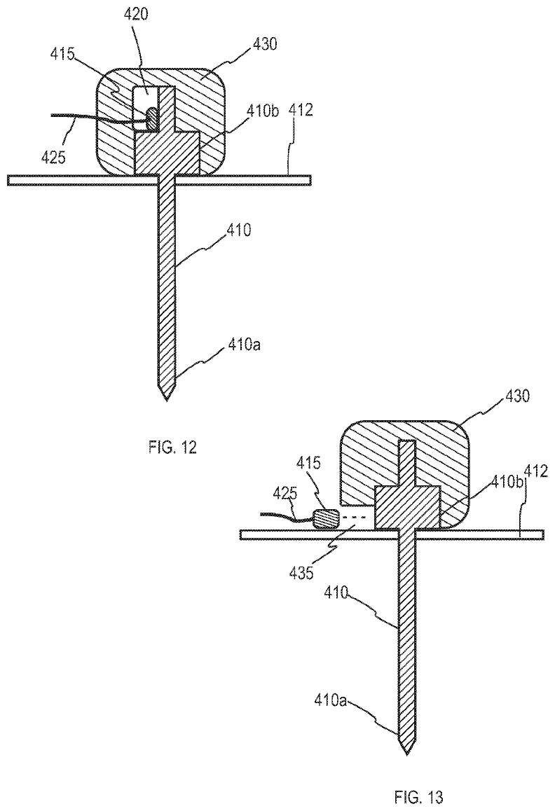

As shown in FIG. 13, a thermal insulation cap (430) has been added to the embodiment of FIG. 11 for the same reasons as described for FIG. 12. The insulation cap (430) in the FIG. 13 embodiment is modified to include a window (435) so as to expose the surface of the needle expanded portion (410b) to the infrared sensor (415).

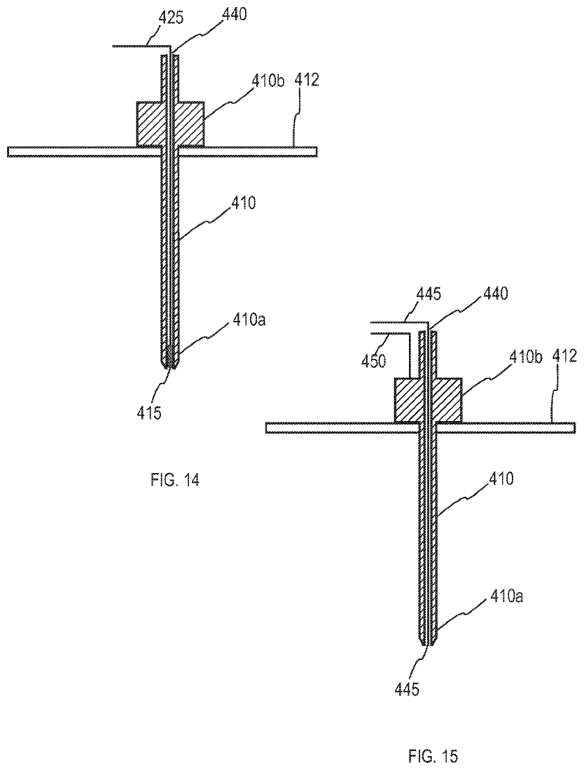

In the embodiment of FIG. 14, the needle (410) has a lumen (440) which receives the temperature sensor (415) and carries the output wires (425) from the sensor (415) to external the needle. In this embodiment, the temperature sensor (415) may directly contact a biological fluid of the subject's subcutaneous tissue at the same tissue depth as the aptamers, thereby providing a very accurate reading that is little affected by the thermal mass of other components of the electrochemical sensor. Moreover, thermal energy is transmitted through the needle (410) wall to the lateral faces of the temperature sensor (415), even further improving the accuracy of the reading.

It may be necessary to increase the cross-sectional area of the needle (410) to accommodate the temperature sensor (415).

It is contemplated that the needle (410) may form part of a thermocouple, as shown in the embodiment of FIG. 15. As is known, a thermocouple is formed by the formation of a junction between two dissimilar metals often by welding. The needle (410) may provide one of the two metals, and the second of the two metals may be a thermocouple grade wire (445) extending through the lumen (440) all the way to the needle (410) tip. The terminus of the thermocouple grade wire (445) is welded to the needle (410) tip. A second wire (450) is soldered to the needle (410), with the thermocouple grade wire (445) and second wire (450) carrying signal to the processor.

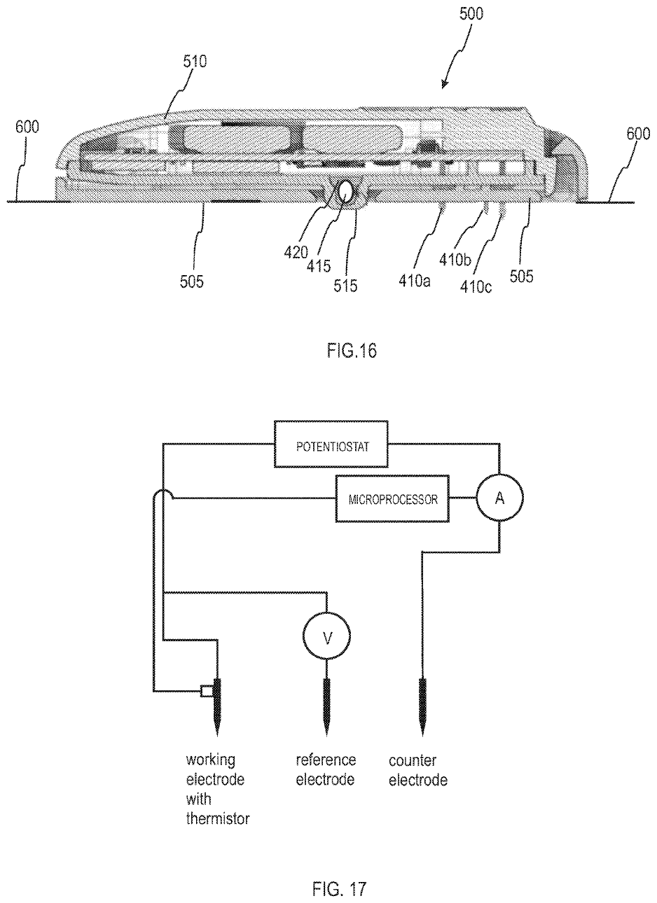

In other embodiments, the temperature sensor is disposed adjacent to the needle so as to determine the temperature of the tissue very proximal to the tissue in which the needle is inserted. Reference is made to FIG. 16 illustrating a unitary, self-contained, wearable apparatus (500) comprising three needles (410a, 410b, 410c) configured as working, counter and reference electrodes, respectively. The needles (410a, 410b, 410c) extend through a skin contacting plate (505) which presents a downwardly facing surface to contact the subject's skin surface (600) when in use. The skin contacting plate (505) additionally provides a lower housing portion which engages an upper housing portion (510) to fully enclose all electronics and other componentry.

The apparatus (500) comprises a pocket (515) dimensioned to receive a temperature sensor (415) such as a thermistor or a thermocouple. The pocket (515) is fabricated from a thin sheet-like material of a thermally conductive plastic, such as a plastic having a metal or other filler to facilitate transmittance of thermal energy from the underlying skin (600) to the temperature sensor (415). The temperature sensor (415) is surrounded by a thermally conductive paste to facilitate transfer of thermal energy from the pocket (515) wall to the temperature sensor (415).

It will be noted that the floor of the pocket (515) extends slightly past the lower surface of the skin contact plate (505). The effect of this arrangement is that the floor of pocket (515) is pushed gently onto the skin surface (600) when the plate (505) is applied thereto, thereby facilitating transfer of thermal energy from the skin (600) to the temperature sensor (415). It will be understood that more firm pushing of the pocket (515) floor onto the skin surface may force blood out the skin capillaries thereby artificially cooling the skin surface (600).

Preferably, only the floor of the pocket (515) is fabricated from a thermally conductive material, with the remainder being fabricated from a material of low thermal conductivity. By that arrangement, thermal energy from the skin will not be routed away from the temperature sensor.

An insulating material (not drawn) may be included to form a ceiling of the pocket (515) to ensure thermal energy is retained about the temperature sensor (415) and not lost to the internal cavity of the housing (505, 510).

The embodiment of FIG. 16 may be modified such that the temperature sensor (415) extends through an aperture in the pocket (515) floor so as to directly contact the skin surface (600). A temperature that is closer to the actual skin temperature would be expected given that thermal energy is not required to traverse any intervening material.

In a further modification, the temperature sensor (415) is an infrared sensor module, and in which case the material of at least the pocket (515) floor should not substantively interfere with its operation. It is contemplated that an aperture could made in the floor to allow the infrared sensor module (415) direct exposure to the skin surface (200) to effect an accurate reading of skin temperature.

Reference is made to FIG. 17, showing a basic circuit for operation of the present invention. The thermistor temperature sensor is shown applied to the working electrode, although it will be understood that it may be applied alternatively to the reference electrode or the counter electrode. In some embodiments, a dedicated needle is provided only for the purpose of temperature measurement.

The preferred embodiments are described herein by reference to needles only, which may interchangeably be a microneedle or the like. However, a wire may be used in place of a needle, in which case the wire may coil around the temperature sensor so as to facilitate transfer of thermal energy thereto. Copper wire would be preferred given the superior thermal conductivity of that metal and the ready availability thereof. It may be necessary to use an introducer (such as a small cannula) to embed the wire within the subcutaneous tissue or biological fluid.

It is understood that the invention comprises all aforementioned variations and modifications and indeed further variations and modifications which fall within the spirit and scope of the present invention.

Accordingly, the spirit and scope of the present invention is not to be limited by the foregoing examples, but is to be understood in the broadest sense allowable by law.

1. An electrochemical sensor apparatus for contacting one or more projecting portions to a biological fluid or a tissue beneath the skin of a subject for an extended period, the apparatus comprising:
one or more projecting portions, each configured to penetrate the skin;
a skin contacting portion defining a skin contacting surface and one or more spaces allowing the one or more projecting portions to extend therethrough;
a movable portion configured to move the one or more projecting portions from a first position behind the skin contacting surface to a second position proud of the skin contacting surface;
optionally a retaining portion configured to, in use, retain the skin contacting surface in contact with the skin; and
a thermal energy sensor configured to determine a temperature of the biological fluid or the tissue about the one or more projecting portions when contacting the biological fluid or the tissue,
wherein the movable portion is configured to move from the first position to the second position.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. The apparatus of claim 1, wherein the one or more projecting portions have an analyte detecting element associated therewith.

47. (canceled)

48. (canceled)

49. (canceled)

50. The apparatus of claim 46, wherein the one or more projecting portions are dedicated to function to determine the temperature of the biological fluid or the tissue about the one or more projecting portions when inserted into the skin of the subject.

51. The apparatus of claim 46, comprising a thermal insulating material configured to retain thermal energy within the one or more projecting portions.

52. The apparatus of claim 51, wherein the thermal insulating material surrounds a terminal portion of the one or more projecting portions that is distal to a portion of one of the one or more projecting portions that inserts into the skin.

53. The apparatus of claim 51, wherein the thermal insulating material forms a cap on a terminal portion of the one or more projecting portions that is distal to a portion of one of the one or more projecting portions that inserts into the skin.

54. The apparatus of claim 51, wherein the thermal insulating material surrounds the thermal energy sensor and the one or more projecting portions.

55. The apparatus of claim 46, wherein the one or more projecting portions are fabricated at least in part from a metal, a metal alloy, a combination of metals, or a ceramic.

56. The apparatus of claim 55, wherein the metal, the metal alloy, a metal in the combination of metals, or the ceramic, has a thermal conductivity k of at least about 200, 300, or 400 W/mK.

57. (canceled)

58. The apparatus of claim 46, wherein the thermal energy sensor is configured to detect thermal energy in one of the one or more projecting portions.

59. The apparatus of claim 46, wherein the thermal energy sensor is applied to or directed toward the one or more projecting portions.

60. The apparatus of claim 46, wherein the thermal energy sensor contacts the one or more projecting portions, or is otherwise in thermal communication therewith.

61. The apparatus of claim 60, wherein the thermal communication is via a thermally conductive flowable substance disposed between the thermal energy sensor and one of the one or more projecting portions.

62. The apparatus of any one of claim 46, wherein the thermal energy sensor is a thermocouple, a thermistor, or an infrared sensor.

63. The apparatus of claim 62, wherein one of the one or more projecting portions forms part of the thermocouple or the thermistor.

64. (canceled)

65. (canceled)

66. The apparatus of claim 65, wherein the lumen holds a thermally conductive flowable substance to form thermal communication between the thermal energy sensor and the surface of the lumen.

67. (canceled)

68. (canceled)

69. The apparatus of claim 67, wherein the thermal energy sensor contacts the skin or is otherwise in thermal communication with the skin.

70. The apparatus of claim 69, wherein the thermal communication is via a thermally conductive solid material that on a first side is in thermal communication with the thermal energy sensor and on a second side is in thermal communication with the skin.

71. (canceled)

72. (canceled)

73. The apparatus of claim 70, wherein the thermally conductive solid material has a thickness of less than about 5 mm, 4 mm, 3 mm, 2 mm, 1 mm, 0.9 mm, 0.8 mm, 0.7 mm, 0.6 mm, 0.5 mm, 0.4 mm, 0.3 mm, 0.2 mm, or 0.1 mm.

74. (canceled)

75. The apparatus of claim 46, wherein the one or more projecting portions and the thermal energy sensor are contained within, or otherwise associated with a housing.

76. (canceled)

77. (canceled)

78. (canceled)

79. (canceled)

80. (canceled)

81. (canceled)

82. (canceled)

83. (canceled)

84. (canceled)

85. (canceled)
